(19) United States
(12) Patent Application Publication
(10) Pub. No.: US 2025/0289807 A1
(43) Pub. Date: Sep. 18, 2025

(54) Inhibitors of ROCK2

(71) Applicant: Graviton Bioscience BV, Amsterdam (NL)

(72) Inventors: Samuel D. Waksal, New York, NY (US); Alexandra Zanin-Zhorov, Staten Island, NY (US); Georgiy Kachkovskyi, Krakow (PL); Magdalena Soluch, Krakow (PL); Anna Jedrzejczak, Krakow (PL); Martin Klvana, Krakow (PL); Dominik Domagala, Krakow (PL)

(21) Appl. No.: 18/861,104

(22) PCT Filed: Apr. 29, 2023
(86) PCT No.: PCT/IB2023/054494

§ 371 Date: Oct. 28, 2024

Related U.S. Application Data
(60) Provisional application No. 63/336,667, filed on Apr. 29, 2022.

(51) Int. Cl. C07D 403/14; A61K 31/506; C07D 405/14; C07D 409/14; C07D 495/04

(52) U.S. Cl. CPC C07D 403/14; A61K 31/506; C07D 405/14; C07D 409/14; C07D 495/04

(57) ABSTRACT

The present disclosure relates to inhibitors of Rho-associated protein kinase (ROCK), pharmaceutical compositions comprising the same, and use thereof for the prevention or treatment of a disease mediated by the ROCK. Particularly, the inhibitors of ROCK are selective for the inhibition of ROCK2.

### FIELD

The present disclosure relates to inhibitors of Rho-associated protein kinase (ROCK), pharmaceutical compositions comprising the same, and use thereof for the prevention or treatment of a disease mediated by the ROCK. Particularly, the inhibitors of ROCK are selective for the inhibition of ROCK2.

### BACKGROUND

Rho-associated coiled-coil kinase (ROCK) is a serine/threonine kinase from the AGC (PKA, PKG, and PKC) kinase family and comprises two isoforms, ROCK1 and ROCK2. The two isoforms are expressed and regulated differently in specific tissues. For example, ROCK1 is ubiquitously expressed at a relatively high level, while ROCK2 is preferentially expressed in certain tissues including heart, brain and skeletal muscle. ROCK is a target of the small GTPase Rho and is involved in diverse cellular activities achieved by phosphorylating downstream effector proteins (MLC, LIMK, ERM, MARCKS, CRMP-2, etc.). Studies have shown that various diseases (e.g., pulmonary fibrosis, cardiac-cerebral vascular disease, neurological disease, cancer, etc.) are related to the pathways mediated by ROCK. As such, ROCK has been considered as an important target in the development of novel drugs.

### SUMMARY

In one aspect, the disclosure provides inhibitors of ROCK2 having the formula I:

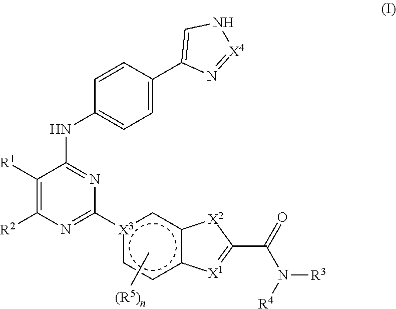

or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
X⁴ is N or CH;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-N¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CR⁶, NR⁷, O and S;
X³ is selected from the group consisting of C, CH and N;
   each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
   each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

In another aspect, the disclosure provides method for the treatment of a disease or disorder mediated by ROCK2, wherein the method comprises administering to a subject in need thereof an effective amount of ROCK 2 inhibitor provided herein or a pharmaceutically acceptable salt thereof.

In embodiments provided in this disclosure, the methods are used to treat a disease or disorder selected from the group consisting of fibrotic diseases, inflammatory diseases, autoimmune diseases, cardiovascular disorders, central nervous system disorders, neoplastic diseases, metabolic syndromes, ocular diseases, renal diseases, pulmonary diseases, muscular dystrophy, sickle cell disease, and viral diseases.

### BRIEF DESCRIPTION OF THE FIGURES

FIG. 1 shows the structure of compounds according to this disclosure and their properties.

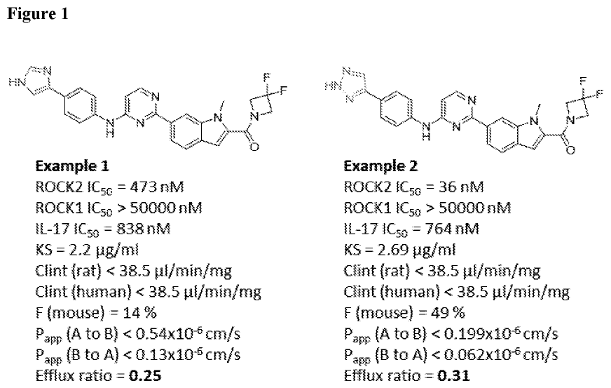

FIGS. 2A-2B shows that the compounds of Example 1 (2A) and Example 2 (2B) inhibit IL-17 in human CD4+ T cells stimulated with Th17-skewing conditions.

FIG. 3 shows that the compounds of Example 1 and Example 2 down-regulate STAT3 phosphorylation induced by Th17-skewing activation in human CD4+ T cells.

FIG. 4 shows that the compounds of Example 1 and Example 2 down-regulate pCofilin in human CD4+ T cells.

FIG. 5 shows that the compounds of Example 1 and Example 2 reduce pro-fibrogenic gene expression in human lung fibroblast MRC-5 cells.

FIG. 6 shows that the compounds of Example 1 and Example 2 down-regulate Collagen Type 1 secretion in human lung fibroblast MRC-5 cells.

FIG. 7 shows that the compounds of Example 1 and Example 2 down-regulate adipogenesis in human adipocytes.

### DETAILED DESCRIPTION

The present invention will now be further described. In the following passages, different aspects of the invention are provided. Each aspect presented may be combined with any other aspect or aspects unless clearly indicated to the contrary. In particular, any feature indicated as being preferred or advantageous may be combined with any other feature or features indicated as being preferred or advantageous.

The compounds, compositions and methods described herein provide selective inhibitors of Rho-associated coiled-coil kinase 2 (ROCK2) for use in the treatment of diseases or disorders, including fibrotic diseases, inflammatory diseases, autoimmune diseases, cardiovascular disorders, central nervous system disorders, neoplastic diseases, metabolic syndromes, ocular diseases, renal diseases, pulmonary diseases, muscular dystrophy, sickle cell disease, and viral diseases.

The compounds for use in the methods and compositions disclosed herein are ROCK inhibitors, and in particular ROCK2 selective inhibitors. The compounds provide excellent inhibitory activity of ROCK (preferably ROCK2) and good selectivity (higher selectivity towards ROCK2 as compared with ROCK1). The compounds may additionally provide one or more of good physicochemical properties (e.g., solubility, physical and/or chemical stability), improved pharmacokinetic properties (e.g., improved bioavailability, proper half-life and duration of action), and improved safety (low toxicity and/or less side effects, wide therapeutic window). Particularly, the ROCK2 inhibitors provided herein may show improved solubility and/or improved bioavailability when administered orally.

In one aspect, this disclosure provides inhibitors of ROCK2 having the formula I:

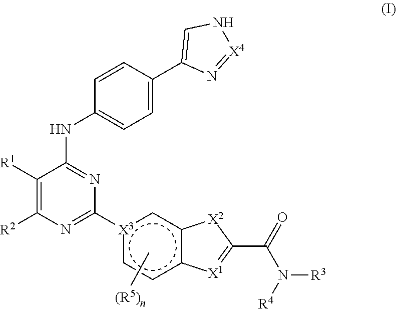

or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
X⁴ is N or CH;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-N¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CHR⁶, NR⁷, O and S;
X³ is selected from the group consisting of C, CH and N;
   each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
   each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

In some embodiments, this disclosure provides inhibitors of ROCK2 having the formula II:

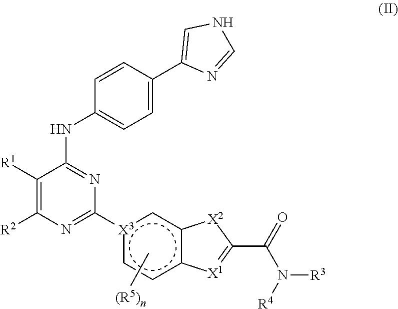

or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-N¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CR⁶, NR⁷, O and S;
X³ is selected from the group consisting of C, CH and N;
   each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
   each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

In some embodiments, this disclosure provides inhibitors of ROCK2 having the formula IIa:

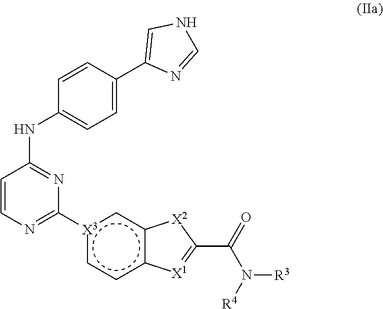

or a pharmaceutically acceptable salt thereof, wherein:
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
X¹ is selected from the group consisting of CR⁶ and N;
X² is selected from the group consisting of CHR⁶, NR⁷, O and S;
X³ is selected from the group consisting of C, CH and N;
   each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
   each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

In some embodiments, this disclosure provides inhibitors of ROCK2 having the formula III:

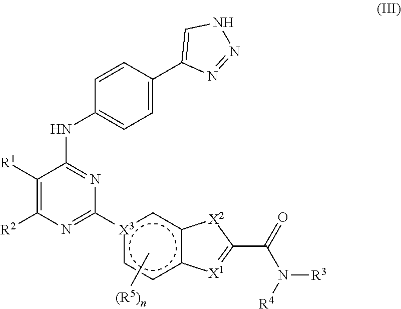

or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring to heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-N¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CR⁶, NR⁷, O and S;
X³ is selected from the group consisting of C, CH and N;
   each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

In some embodiments, this disclosure provides inhibitors of ROCK2 having the formula IIIa:

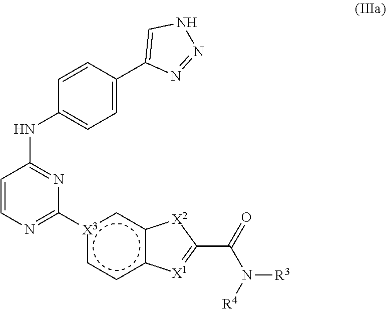

or a pharmaceutically acceptable salt thereof, wherein:
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
X¹ is selected from the group consisting of CR⁶ and N;
X² is selected from the group consisting of CR⁶, NR⁷, O and S;
X³ is selected from the group consisting of C, CH and N;
   each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
   each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

For each of the formulas of inhibitors of ROCK2 provided herein, in the partial structures:

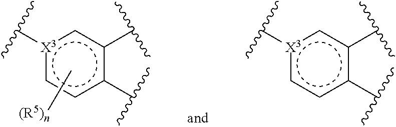

the dash circle represents one or more optional double bonds. Accordingly, this partial structure may be unsaturated, have a single double bond at any chemically allowed position, have two double bonds at chemically allowed positions, or be an aromatic ring system. As would be understood by a person skilled in the art, the double bonds in such a ring system will not be at directly adjacent positions (i.e., they do not share a carbon atom). In embodiments, partial structure includes:

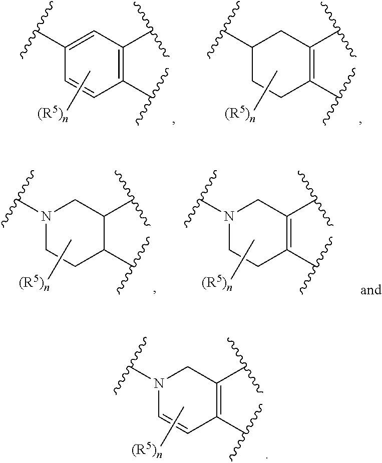

In other embodiments, the partial structure includes:

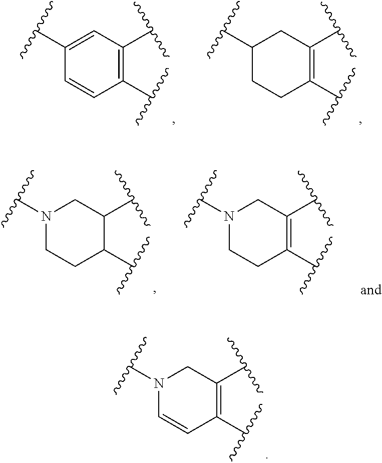 [SMILES: *c1ccc(*)c(*)c1 ; *C1=C(*)CC(*)CC1 ; *C1CCN(*)CC1* ; *C1=C(*)CN(*)CC1 ; *C1=C(*)CN(*)C=C1]

The term “heteroatom” as used herein means an atom of any element other than carbon or hydrogen. Preferred heteroatoms are nitrogen, oxygen, and sulfur.

The term “halogen” or “halo” designates —F, —Cl, —Br or —I. Preferred halo gens are —F, —Cl and —Br.

The term “hydroxyl” means —OH.

The term “oxo” as used herein refers to an oxygen atom that has a double bond to another atom (i.e., the substituent ═O), particularly to carbon.

The term “alkyl” refers to the radical of saturated aliphatic groups, including straight-chain alkyl groups and branched-chain alkyl groups. Accordingly, C₁-C₆ alkyl includes methyl, ethyl, n-propyl, isopropyl, n-butyl, sec-butyl, iso-butyl, tert-butyl, n-pentyl, and the like.

The term “cycloalkyl” refers to saturated, carbocyclic groups having from 3 to 7 carbons in the ring. Cycloalkyl groups include cyclopropyl, cyclobutyl, cyclopentyl and cyclohexyl.

The term “alkenyl” refers to a linear or branched hydrocarbyl having a double bond and 2-6 carbon atoms (“C₂-C₆ alkenyl”). The alkenyl includes vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, and the like. When the compound of the present disclosure contains an alkenyl group, the compound may exist as the E-form, the Z-form, or any mixture thereof.

The term “alkynyl” refers to a linear or branched hydrocarbyl having a triple bond and 2-6 carbon atoms (“C₂-C₆ alkynyl). The alkynyl includes ethynyl, propynyl, and the like.

The term “aryl” as used herein includes 5- and 6-membered single-ring aromatic groups that may include from zero to four heteroatoms, for example, benzene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaromatics” or “heteroaryl”. The term “aryl” also includes 7- to 14-membered polycyclic ring systems having two or more cyclic rings in which two or more carbons are common to two adjoining rings (the rings are “fused rings”) wherein at least one of the rings is aromatic (including heteroaryl), e.g., the other cyclic rings can be fused cycloalkyls, cycloalkenyls, aryls, heteroaryl and/or heterocyclic groups. Single-ring heteroaryl groups may have from 1 to 3 ring heteroatoms and fused polycyclic heteroaryl groups may have from 1 to 5 ring heteroatoms, wherein the ring heteroatoms are selected from N, O and S.

The terms “heterocyclyl” or “heterocyclic group” refer to 3- to 10-membered ring structures, more preferably 5- or 6-membered rings, whose ring structures include one to four heteroatoms. Heterocycles can also be polycycles. Heterocyclic groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like.

The term “aralkyl”, as used herein, refers to a C₁-C₆ alkyl group substituted with an aryl group (e.g., an aromatic or heteroaromatic group).

As used herein, the definition of each expression, e.g. alkyl, m, n, R, etc., when it occurs more than once in any structure, is intended to be independent of its definition elsewhere in the same structure.

It will be understood that “substituted”, “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

Certain compounds provided in this disclosure may exist in particular geometric or stereoisomeric forms. The disclosure contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

The term “pharmaceutically-acceptable salts” refers to the relatively non-toxic, inorganic and organic acid addition salts of compounds disclosed herein and inorganic and organic basic addition salts of the compounds disclosed herein.

As set out above, certain embodiments of the ROCK2 inhibitors may contain a basic functional group, such as amino, and are capable of forming pharmaceutically-acceptable salts with pharmaceutically-acceptable acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds provided in this disclosure may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

The ROCK2 inhibitors provided herein may show improved solubility and/or improved bioavailability when administered orally. For example, the compounds of Examples 1 and 2 show kinetic solubilities of 2.2 μg/ml and 2.69 μg/ml, respectively, which is significantly improved as compared to comparative compound A, having a kinetic solubility of <1.9 μg/ml.

### Method of Treatment

The present disclosure provides a method for the prevention or treatment of a disease mediated by ROCK2, wherein the method comprises administering to a subject in need thereof an effective amount of a ROCK2 inhibitor as disclosed herein or a pharmaceutically acceptable salt thereof.

In some embodiments, the present disclosure provides methods for the treatment of at least one disease or disorder selected from the group comprising fibrotic diseases, inflammatory diseases, and autoimmune diseases, the method comprising administering to a subject in need thereof a therapeutically effective amount of a compound or a composition as defined herein.

In other embodiments, the disclosure provides methods for the treatment of a cardiovascular disorder, a central nervous system disorder, a neoplastic disease, or a metabolic syndrome, the method comprising administering to a subject in need thereof a therapeutically effective amount of a compound or a composition as defined herein.

In some embodiments, the disease mediated by ROCK2 is an autoimmune disorder including rheumatoid arthritis, systemic lupus erythematosus (SLE; lupus), psoriasis, psoriatic arthritis, multiple sclerosis, Crohn's disease, ulcerative colitis, atopic dermatitis, eczema, or graft-versus-host disease (GVHD; acute and chronic), idiopathic pulmonary fibrosis and scleroderma.

Other autoimmune disorders that may be treated according to the methods provided in this disclosure include acute disseminated encephalomyelitis (ADEM), acute necrotizing hemorrhagic leukoencephalitis, Addison's disease, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thyroid disease, autoimmune urticaria, axonal & neuronal neuropathies, Balo disease, Behcet's disease, bullous pemphigoid, cardiomyopathy, Castleman disease, Celiac disease, Chagas disease, chronic inflammatory demyelinating polyneuropathy (CIDP), chronic recurrent multifocal ostomyelitis (CRMO), Churg-Strauss syndrome, Cogans syndrome, coxsackie myocarditis, CREST disease, demyelinating neuropathies, dermatitis herpetiformis, dermatomyositis, Devic's disease (neuromyelitis optica), discoid lupus, Dressler's syndrome, eosinophilic esophagitis, eosinophilic fasciitis, erythema nodosum, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, Hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile myositis, Kawasaki syndrome, Lambert-Eaton syndrome, Leukocytoclastic vasculitis, lichen planus, lichen sclerosus, ligneous conjunctivitis, Linear IgA disease (LAD), Meniere's disease, microscopic polyangiitis, mixed connective tissue disease (MCTD), Mooren's ulcer, Mucha-Habermann disease, myasthenia gravis, myositis, neuromyelitis optica, neutropenia, ocular cicatricial pemphigoid, optic neuritis, palindromic rheumatism, pediatric autoimmune neuropsychiatric disorders associated with Streptococcus (PANDAS), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, autoimmune polyglandular syndromes (type I, II, III), polymyalgia rheumatica, polymyositis, post myocardial infarction syndrome, post pericardiotomy syndrome, progesterone dermatitis, primary biliary cirrhosis, primary sclerosing cholangitis, psoriatic arthritis, pyoderma gangrenosum, pure red cell aplasia, Raynaud's phenomenon, reactive arthritis, reflex sympathetic dystrophy, Reiter's syndrome, relapsing polychondritis, retroperitoneal fibrosis, sarcoidosis, Schmidt syndrome, scleritis, Sjogren's syndrome, Sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, Transverse myelitis, undifferentiated connective tissue disease (UCTD), type-1 autoimmune diabetes, uveitis, vasculitis, vesiculobullous dermatosis, vitiligo, and Wegener's granulomatosis (granulomatosis with polyangiitis; GPA).

Inflammatory disorders that can be treated by methods provided in this disclosure include, but are not limited to, cardiovascular inflammation, pulmonary inflammation, renal inflammation, arteriosclerosis and sepsis.

Fibrotic disorders that can be treated by methods provided in this disclosure include idiopathic pulmonary fibrosis, renal fibrosis, kidney fibrosis, ocular fibrosis, cardiac fibrosis, NASH, scleroderma, systemic sclerosis, and cirrhosis.

In another embodiment, the disclosure provides a method for the treatment of muscular dystrophy (Duchenne muscular dystrophy). In another embodiment, the disclosure provides a method for the treatment of myotonic dystrophy.

In other embodiments, the ROCK2 inhibitors provided herein may be used to inhibit tumor cell growth and metastasis, and angiogenesis, and are useful for treating neoplastic diseases. Neoplastic diseases include any malignant growth or tumor caused by abnormal or uncontrolled cell division. Neoplastic diseases include lymphoma, carcinoma, leukemia, sarcoma and blastoma. Non-limiting examples include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, bladder cancer, liver cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

In other embodiments, the ROCK2 inhibitors provided herein may be used in the treatment of a cardiovascular disorder including hypertension, cardiomyopathy, cardiac remodeling, atherosclerosis, restenosis, cardiac hypertrophy, cerebral ischemia, cerebral vasospasm, and erectile dysfunction.

In other embodiments, the ROCK2 inhibitors provided herein may be used in the treatment of a pulmonary disorder including idiopathic pulmonary fibrosis, chronic obstructive pulmonary disease, and asthma.

In other embodiments, the ROCK2 inhibitors provided herein may be used in the treatment of a central nervous system disorder, including neuronal degeneration or spinal cord injury, traumatic brain injury, cerebral cavernous malformation, Huntington's disease, Parkinson's disease, Alzheimer's disease, Amyotrophic lateral sclerosis (ALS), or multiple sclerosis.

In other embodiments, the disclosure provides methods for the treatment of renal diseases including polycystic kidney disease, renal fibrosis and diabetic renal disease.

In other embodiments, the ROCK2 inhibitors provided herein may be used in the treatment of a metabolic disease including insulin resistance, hyperinsulinemia, type 2 diabetes, obesity, metabolic syndrome and glucose intolerance. The ROCK2 inhibitors may be used to effect weight loss and/or limit weight gain. In an embodiment, a ROCK2 inhibitor is used to reduce or prevent insulin resistance or restore insulin sensitivity.

In other embodiments, the ROCK2 inhibitors provided herein may be used in the treatment of an ocular disorder including ocular hypertension, age related macular degeneration (AMD; wet and dry), choroidal neovascularization (CNV), choroidal tumor, diabetic macular edema (DME), iris neovascularization, uveitis, glaucoma, primary open-angle glaucoma, acute angle-closure glaucoma, pigmentary glaucoma, congenital glaucoma, normal tension glaucoma, secondary glaucoma, neo vascular glaucoma, geographic atrophy, and retinitis of prematurity (ROP).

In another embodiment, the disclosure provides methods for the treatment of sickle cell disease.

In other embodiments, the ROCK2 inhibitors provided herein may be used to treat (i.e., cure or reduce the severity of, etc.) viral infections, particularly coronavirus infections such as SARS-CoV-1, SARS-CoV-2, and MERS-CoV, and to treat or prevent the sequelae resulting from the viral infection, including the coronavirus infection such as SARS-CoV-1, SARS-CoV-2, and MERS-CoV. In some embodiments, the viral infection is a SARS-CoV-1 infection. In some embodiments, the viral infection is a SARS-CoV-2 infection. In some embodiments, the viral infection is a MERS-CoV infection. In embodiments, the sequelae include one or more of the group consisting of fatigue, dyspnea (difficulty breathing), cough, arthralgia (joint pain), myalgia, headache, chest pain, fever, palpitations, myocardial inflammation, ventricular dysfunction, stroke, pulmonary function abnormalities, fibrosis (such as pulmonary fibrosis), renal dysfunction rash, alopecia, olfactory and/or gustatory dysfunction, sleep dysregulation, cognitive impairment altered, memory impairment, depression, anxiety, changes in mood and combinations thereof. In embodiments, the sequelae include inflammation and/or fibrosis.

### Pharmaceutical Compositions

In one aspect, the present disclosure provides pharmaceutically acceptable compositions for use in the treatment of viral diseases which comprise a therapeutically-effective amount of one or more of the ROCK2 inhibitors provided in this disclosure, formulated together with one or more pharmaceutically acceptable carriers. As described below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginal or intrarectal administration, for example, as a suppository, pessary, cream or foam; (5) sublingual administration; (6) ocular administration; (7) transdermal administration; or (8) nasal administration.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), solvent, or solvent encapsulating material, involved in carrying or transporting the subject compound from one organ, or portion of the body, to another organ, or portion of the body. Each carrier should be compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The compounds of this disclosure may be formulated with conventional carriers and excipients, which can be selected in accord with ordinary practice. Tablets can contain excipients, glidants, fillers, binders and the like. Aqueous formulations are prepared in sterile form, and when intended for delivery by other than oral administration generally can be isotonic. All formulations can optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like.

While it is possible for the ROCK2 inhibitors disclosed herein (herein referred to as the “active ingredients”) to be administered alone, it may be preferable to present them as pharmaceutical formulations. The formulations, both for veterinary and for human use, of the disclosure comprise at least one active ingredient, as provided above, together with one or more acceptable carriers therefor and optionally other therapeutic ingredients, particularly those additional therapeutic ingredients as discussed herein.

The formulations include those suitable for the administration routes provided herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the present disclosure suitable for oral administration may be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues e.g. mouth and skin, the formulations are preferably applied as a topical solution, ointment or cream containing the active ingredient(s). The active ingredient may be present in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this disclosure may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulsifying agents and emulsion stabilizers suitable for use in the formulation of the disclosure include Tween® 60, Span® 80, cetostearyl alcohol, benzyl alcohol, myristyl alcohol, glyceryl mono-stearate and sodium lauryl sulfate. Further emulsifying agents and emulsion stabilizers suitable for use in the formulation of the disclosure include Tween® 80.

The choice of suitable oils or fats for the formulation is based on achieving the desired properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present disclosure comprise a combination according to the disclosure together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example starch, mannitol, calcium phosphate or kaolin, or as soft gelatin capsules wherein the active ingredient is mixed with water or an oil medium, such as peanut oil, liquid paraffin or olive oil.

Aqueous suspensions of the disclosure contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin and Captisol (=Sulfobutyl ether beta-cyclodextrin; SEB-beta-CD).

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form can vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient may be present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds.

Formulations suitable for vaginal administration may be presented as suppositories, pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The disclosure further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the disclosure are used to provide controlled release pharmaceutical formulations containing as active ingredient one or more compounds of the disclosure (“controlled release formulations”) in which the release of the active ingredient are controlled and regulated to allow less frequency dosing or to improve the pharmacokinetic or toxicity profile of a given active ingredient.

The patient receiving this treatment is any animal in need, including primates, in particular humans, and other mammals such as horse, cattle, swine and sheep; and poultry and pets in general.

### EXAMPLES

### Example 1

### Method A. Exemplified by Synthesis of 2-[2-(3,3-difluoroazetidine-1-carbonyl)-1-methyl-1H-indol-6-yl]-N-[4-(1H-imidazol-5-yl)phenyl]pyrimidin-4-amine

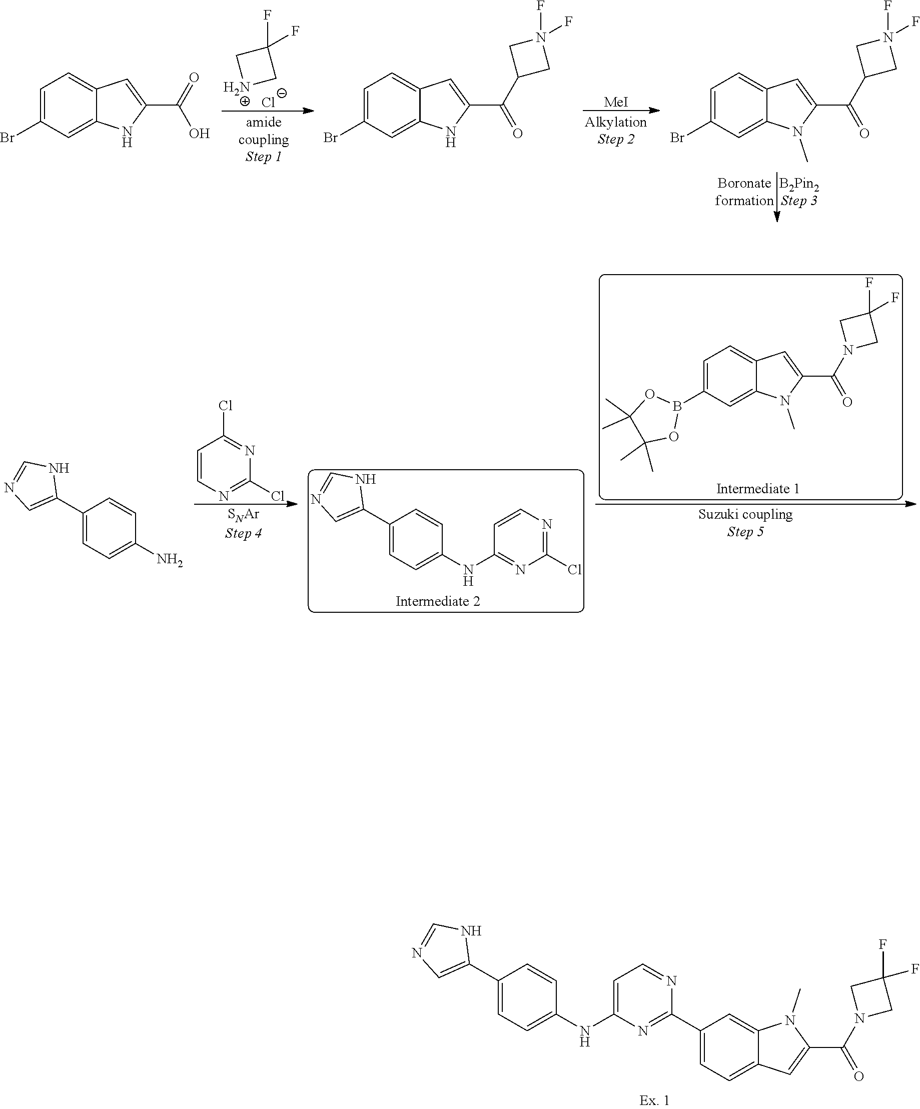

### Step 1: Synthesis of Intermediate 1: 6-bromo-2-(3,3-difluoroazetidine-1-carbonyl)-1H-indole

6-Bromoindole-2-carboxylic acid (5.26 g, 21.91 mmol, 1.00 equiv) was dissolved in DCM anhyd. (25.0 mL), next to the reaction mixture was added Oxalyl chloride (2.04 ml, 24.10 mmol, 1.10 equiv) dropwise at 0° C., followed by addition of a few drops of DMF. The mixture was stirred at 0° C. for 1 h and then the cold-bath was removed and the stirring was continued at room temperature for 3 h. The TLC analysis showed full conversion of starting material, UPLC-MS analysis showed mass corresponds to proper ester (sample quenched with MeOH). The reaction mixture was evaporated to dryness, two time co-evaporated with ACN. The resulting cream colored powder was then redissolved in THF (25.0 mL) and transferred dropwise by syringe to a stirred ice-cold solution of 3,3-Difluoroazetidine hydrochloride (2.84 g, 21.91 mmol, 1.00 equiv) and N,N-Diisopropylethylamine (15.27 mL, 87.65 mmol, 4.00 equiv) in DCM (25.0 mL). After 1 h at 0° C. the cold bath was removed and stirred at room temperature overnight. Then a solution of NaHCO₃ was added and the mixture of DCM and THF were removed in vacuo. The precipitate was collected by vacuum filtration and washed with water and was dried under high vacuum for 18 h to give 6-bromo-2-(3,3-difluoroazetidine-1-carbonyl)-1H-indole (6.5 g, 90% yield). ¹H NMR (400 MHz, DMSO-d₆) δ 11.93-11.86 (m, 1H), 7.65-7.59 (m, 2H), 7.21 (dd, J=8.6, 1.8 Hz, 1H), 6.95 (dd, J=2.2, 0.9 Hz, 1H), 4.77 (m, J=170.3 Hz, 4H). UPLC-MS, m/z: [M+H]=314.8

### Step 2: Synthesis of 6-bromo-2-(3,3-difluoroazetidine-1-carbonyl)-1-methyl-1H-indole

To a solution of 6-bromo-2-(3,3-difluoroazetidine-1-carbonyl)-1H-indole (1.20 g, 3.81 mmol, 1.00 equiv) in DMF anhyd. (15.0 mL) was added NaH (0.12 g, 4.57 mmol, 1.20 equiv) portionwise at 0° C. The resulting mixture was stirred at the same temperature for 1 h. Then Methyl iodide (0.26 mL, 4.19 mmol, 1.10 equiv) was added dropwise. The stirring was continued for 30 min at 0° C. and then at room temperature overnight. Progress of the reaction was monitored using TLC analysis (eluent 10% MeOH-DCM). The reaction mixture was quenched with water and extracted with EtOAc. The combined organic layers were washed with brine, dried over Na₂SO₄, filtered and concentrated to give 6-bromo-2-(3,3-difluoroazetidine-1-carbonyl)-1-methyl-1H-indole (1.09 g, 70% yield) which was used in the next step without purification. ¹H NMR (400 MHz, DMSO-d₆) δ 7.87-7.84 (m, 1H), 7.59 (d, J=8.5 Hz, 1H), 7.25 (dd, J=8.5, 1.7 Hz, 1H), 7.04 (d, J=0.8 Hz, 1H), 5.04-4.38 (m, 4H), 3.93 (s, 3H). UPLC-MS, m/z: [M+H]⁺=330.75

### Step 3: 2-(3,3-difluoroazetidine-1-carbonyl)-1-methyl-6-(4,4,5,5-tetramethyl-1,3,2-dioxaborolan-2-yl)-1H-indole (Intermediate 1)

6-Bromo-2-(3,3-difluoroazetidine-1-carbonyl)-1-methyl-1H-indole (0.74 g, 2.25 mmol, 1.00 equiv), Bis(pinacolato)diboron (0.85 g, 3.37 mmol, 1.50 equiv) and Potassium acetate (0.66 g, 6.74 mmol, 3.00 equiv) were dissolved in Dioxane anhydrous (9.0 mL). The reaction mixture was purged with argon 4 times and then 1,1′-Bis(diphenylphosphino)-ferrocene-palladium(II) (0.05 g, 0.06 mmol, 0.03 equiv) was added. The reaction mixture was placed in the preheated oil bath at 90° C. overnight. The reaction mixture was cooled to room temperature, filtered through Celit pad, concentrated and purified using flash column chromatography (0-10% MeOH in DCM, gradient elution) to give 2-(3,3-difluoroazetidine-1-carbonyl)-1-methyl-6-(4,4,5,5-tetramethyl-1,3,2-dioxaborolan-2-yl)-1H-indole (Intermediate 1) (0.81 g, 89% yield). ¹H NMR (400 MHz, DMSO-d₆) δ 7.83 (d, J=0.9 Hz, 1H), 7.63 (dd, J=8.0, 0.8 Hz, 1H), 7.42 (dd, J=8.0, 0.9 Hz, 1H), 7.03 (d, J=0.9 Hz, 1H), 4.70 (d, J=105.5 Hz, 5H), 3.98 (s, 3H), 1.33 (s, 11H). UPLC-MS, m/z: [M+H]⁺=377.15.

### Step 4: Synthesis of Intermediate 2: 2-chloro-N-[4-(1H-imidazol-5-yl)phenyl]pyrimidin-4-amine

To a solution of 4-(1H-imidazol-5-yl)aniline (0.89 g, 5.60 mmol, 1.00 equiv) in Ethanol (9.0 mL) was added N,N-Diisopropylethylamine (DIPEA) (1.46 mL, 8.38 mmol, 1.50 equiv) and the reaction mixture was stirred for 15 min at room temperature. Then 2,4-Dichloropyrimidine (0.83 g, 5.6 mmol, 1.00 equiv) was added and the reaction mixture was allowed to stir for 24 h. It was filtered. The obtained solid cake was redissolved in boiled Ethanol and filtered again and dried under high vacuum to give 2-chloro-N-[4-(1H-imidazol-5-yl)phenyl]pyrimidin-4-amine (0.89 g, 56% yield). ¹H NMR (300 MHz, DMSO-d6) δ 12.90 (s, 1H), 10.15 (s, 1H), 8.14 (d, J=5.9 Hz, 1H), 8.03 (s, 2H), 7.60 (d, J=2.4 Hz, 4H), 6.78 (d, J=5.9 Hz, 1H). UPLC-MS, m/z: [M+H]⁺=271.95.

### Step 5: 2-[2-(3,3-difluoroazetidine-1-carbonyl)-1-methyl-1H-indol-6-yl]-N-[4-(1H-imidazol-5-yl)phenyl]pyrimidin-4-amine

A mixture of Intermediate 1 (0.20 g, 0.49 mmol, 1.40 equiv), Intermediate 2 (0.10 g, 0.35 mmol, 1.00 equiv) and Sodium carbonate (0.07 g, 0.70 mmol, 2.00 equiv) was dissolved in a mixture of Dimetoxyethane (6.0 mL) and Water (2.0 mL) and degassed with argon for 15 min. Then, Tetrakis(triphenylphosphine)palladium (0.024 g, 0.021 mmol, 0.06 equiv) was added and the reaction mixture was stirred at 110° C. in microwave reactor. The progress of the reaction was monitored using UPLC-MS analysis, the full conversion of starting materials was obtained after 4 h of stirring. After cooling, the mixture was diluted with EtOAc and filtered through Celit pad. The filtrate was concentrated in vacuo and purified via preparative HPLC (Mobile phase: H₂O+0.1% FA, ACN+0.1% FA Column C18 prep) to provide compound 2-[2-(3,3-difluoroazetidine-1-carbonyl)-1-methyl-1H-indol-6-yl]-N-[4-(1H-imidazol-5-yl)phenyl]pyrimidin-4-amine, formic salt (0.060 g, 35%) as a yellow foam. ¹H NMR (400 MHz, DMSO-d6) δ 9.69 (s, 1H), 8.56 (s, 1H), 8.41 (d, J=5.8 Hz, 1H), 8.28 (d, J=7.2 Hz, 2H), 8.21 (dd, J=8.5, 1.4 Hz, 1H), 7.81 (s, 4H), 7.75 (d, J=8.5 Hz, 1H), 7.71 (d, J=1.1 Hz, 1H), 7.55 (s, 1H), 7.08 (s, 1H), 6.73 (d, J=5.8 Hz, 1H), 4.73 (d, J=111.7 Hz, 4H), 4.05 (s, 3H). LCMS, m/z: [M+H]⁺=486.41.

### Example 2

### Method B—Exemplified by Synthesis of 2-[2-(3,3-difluoroazetidine-1-carbonyl)-1-methyl-1H-indol-6-yl]-N-[4-(1H-1,2,3-triazol-4-yl)phenyl]pyrimidin-4-amine

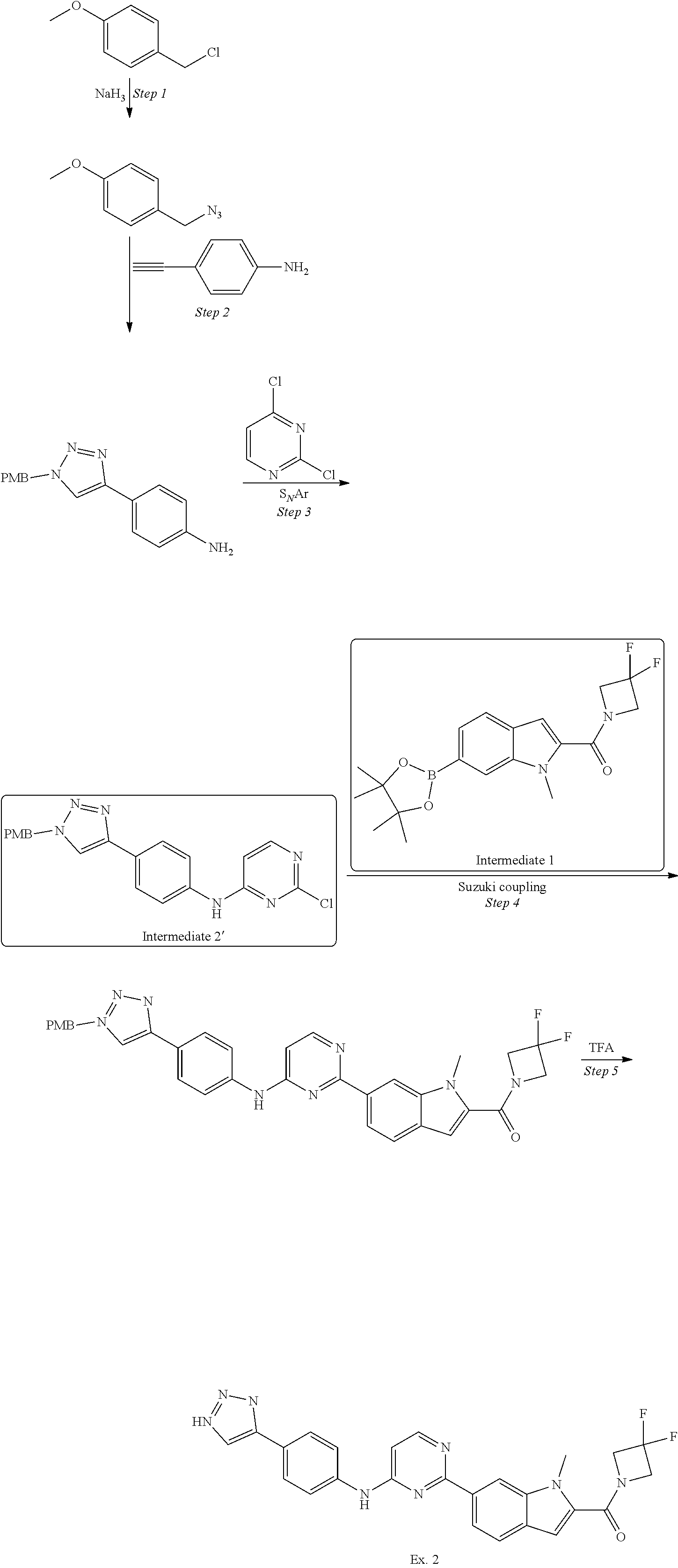

### Step 1′: Synthesis of Intermediate 2′: 1-(azidomethyl)-4-methoxybenzene

A mixture of 4-Methoxybenzyl chloride (2.00 g, 12.77 mmol, 1.00 equiv) and Sodium azide (0.99 g, 15.32 mmol, 1.20 equiv) in Dimethyl sulfoxide anhydrous (20.0 mL) was stirred at room temperature overnight. The progress of the reaction was monitored by TLC analysis (eluent: 9:1 DCM:MeOH). The mixture was portioned between water and EtOAc. The aqueous phase was extracted with EtOAc. The combined organic layers were washed with brine, dried over Na₂SO₄, filtered and evaporated to dryness to obtain 1-(azidomethyl)-4-methoxybenzene (1.98 g, 94%) as a yellowish liquid.

### Step 2′: Synthesis of 4-{1-[(4-methoxyphenyl)methyl]-1H-1,2,3-triazol-4-yl}aniline

To a mixture of 1-(azidomethyl)-4-methoxybenzene (1.98 g, 12.013 mmol, 1.0 equiv) and 4-Ethynylaniline (1.40 g, 12.00 mmol, 1.00 equiv) in Dimethyl sulfoxide anhydrous (29.70 mL) Copper(II) sulfate pentahydrate (0.60 g, 2.40 mmol, 0.20 equiv) was added followed by Sodium ascorbate (0.92 g, 4.80 mmol, 0.40 equiv) and the resulting mixture was stirred at rt for 2 h. Full conversion of starting materials was observed according to UPLC-MS analysis. The mixture was poured into cold water. The precipitated solid was filtered, washed with water and dried to obtain 4-{1-[(4-methoxyphenyl)methyl]-1H-1,2,3-triazol-4-yl}aniline (2.5 g, 73%) as a yellow solid. ¹H NMR (300 MHz, DMSO-d6) δ 8.26 (s, 1H), 7.48 (d, J=8.0 Hz, 2H), 7.37-7.23 (m, 2H), 7.00-6.87 (m, 2H), 6.58 (d, J=8.0 Hz, 2H), 5.49 (s, 2H), 5.19 (s, 2H), 3.74 (s, 3H). UPLC-MS, m/z: [M+H]⁺=281.10.

### Step 3′: Synthesis of 2-chloro-N-(4-{1-[(4-methoxyphenyl)methyl]-1H-1,2,3-triazol-4-yl}phenyl)pyrimidin-4-amine

To a solution of 4-{2-[(4-methoxyphenyl)methyl]-2H-1,2,3-triazol-4-yl}aniline (2.50 g, 8.83 mmol, 1.00 equiv), in Ethanol (15.0 mL) was added N,N-diisopropylethylamine (DIPEA) (1.999 mL, 11.48 mmol, 1.30 equiv) and the reaction mixture was stirred for 15 min at room temperature. Then 2,4-Dichloropyrimidine (1.31 g, 8.83 mmol, 1.00 equiv) was added and the reaction mixture was allowed to stir at 90° C. overnight. It was evaporated to dryness. The crude was purified by FCC (0-5% of MeOH in DCM) to provide 2-chloro-N-(4-{1-[(4-methoxyphenyl)methyl]-1H-1,2,3-triazol-4-yl}phenyl)pyrimidin-4-amine (1.43 g, 40%) as a yellow solid. ¹H NMR (300 MHz, DMSO-d6) δ 10.13 (s, 1H), 8.52 (s, 1H), 8.18 (d, J=5.9 Hz, 1H), 7.83 (d, J=8.6 Hz, 2H), 7.66 (d, J=8.3 Hz, 2H), 7.34 (d, J=8.4 Hz, 2H), 7.01-6.91 (m, 2H), 6.79 (d, J=5.9 Hz, 1H), 5.56 (s, 2H), 3.74 (s, 3H). UPLC-MS, m/z: [M+H]⁻=392.10.

### Step 4′: 2-[2-(3,3-difluoroazetidine-1-carbonyl)-1-methyl-1H-indol-6-yl]-N-(4-{1-[(4-methoxyphenyl)methyl]-1H-1,2,3-triazol-4-yl}phenyl)pyrimidin-4-amine

A mixture of Intermediate 1 (0.35 g, 0.42 mmol, 1.10 equiv), Intermediate 2′ (0.25 g, 0.38 mmol, 1.00 equiv), and Sodium carbonate (0.12 g, 1.14 mmol, 3.00 equiv) was dissolved in a mixture of Dimetoxyethane (6.0 mL) and Water (2.0 mL) and degassed with argon for 10 min. Then, Tetrakis(triphenylphosphine)palladium (0.04 g, 0.04 mmol, 0.10 equiv) was added and the reaction mixture was stirred at 110° C. in microwave reactor. The progress of the reaction was monitored using UPLC-MS analysis, the full conversion of starting materials was obtained after 4 h of stirring. After cooling, the mixture was diluted with EtOAc and filtered through Celit pad. The filtrate was concentrated to dryness to provide crude compound 2-[2-(3,3-difluoroazetidine-1-carbonyl)-1-methyl-1H-indol-6-yl]-N-(4-{1-[(4-methoxyphenyl)methyl]-1H-1,2,3-triazol-4-yl}phenyl)pyrimidin-4-amine (0.2 g) which was used in the next step without further purification. UPLC-MS, m/z: [M=H]⁺=607.10. ¹H NMR analysis was not performed.

### Step 5′: 2-[2-(3,3-difluoroazetidine-1-carbonyl)-1-methyl-1H-indol-6-yl]-N-[4-(1H-1,2,3-triazol-4-yl)phenyl]pyrimidin-4-amine

The 2-[2-(3,3-difluoroazetidine-1-carbonyl)-1-methyl-1H-indol-6-yl]-N-(4-{2-[(4-methoxyphenyl)methyl]-2H-1,2,3-triazol-4-yl}phenyl)pyrimidin-4-amine (0.20 g, 0.20 mmol, 1.00 equiv) was dissolved in Trifluoroacetic acid (1.06 mL, 13.85 mmol, 70.00 equiv) and the reaction mixture was stirred at 100° C. overnight. After that time no partial conversion was observed. The reaction mixture was evaporated to dryness and purified using prep HPLC (Mobile phases: ACN+0.1% FA, H2O+0.1% FA, Column C18 prep) to give 2-[2-(3,3-difluoroazetidine-1-carbonyl)-1-methyl-1H-indol-6-yl]-N-[4-(1H-1,2,3-triazol-4-yl)phenyl]pyrimidin-4-amine (0.025 g, 26%). ¹H NMR (400 MHz, DMSO-d6) δ 9.89 (s, 1H), 8.57 (s, 1H), 8.44 (d, J=5.9 Hz, 1H), 8.32 (d, J=16.4 Hz, 2H), 8.21 (d, J=8.6 Hz, 1H), 7.93 (q, J=8.6 Hz, 4H), 7.76 (d, J=8.6 Hz, 1H), 7.09 (s, 1H), 6.78 (d, J=5.8 Hz, 1H), 4.86 (s, 4H), 4.06 (s, 3H). LCMS, m/z: [M=H]⁺=487.18.

### Example 3

### Method C. Exemplified by Synthesis of 2-[2-(3,3-difluoroazetidine-1-carbonyl)-4H,5H,6H,7H-thieno[2,3-c]pyridin-6-yl]-N-[4-(1H-imidazol-4-yl)phenyl]pyrimidin-4-amine (Ex. 3) and 2-[2-(3,3-difluoroazetidine-1-carbonyl)-4H,5H,6H,7H-thieno[2,3-c]pyridin-6-yl]-N-[4-(1H-1,2,3-triazol-4-yl)phenyl]pyrimidin-4-amine (Ex. 3′)

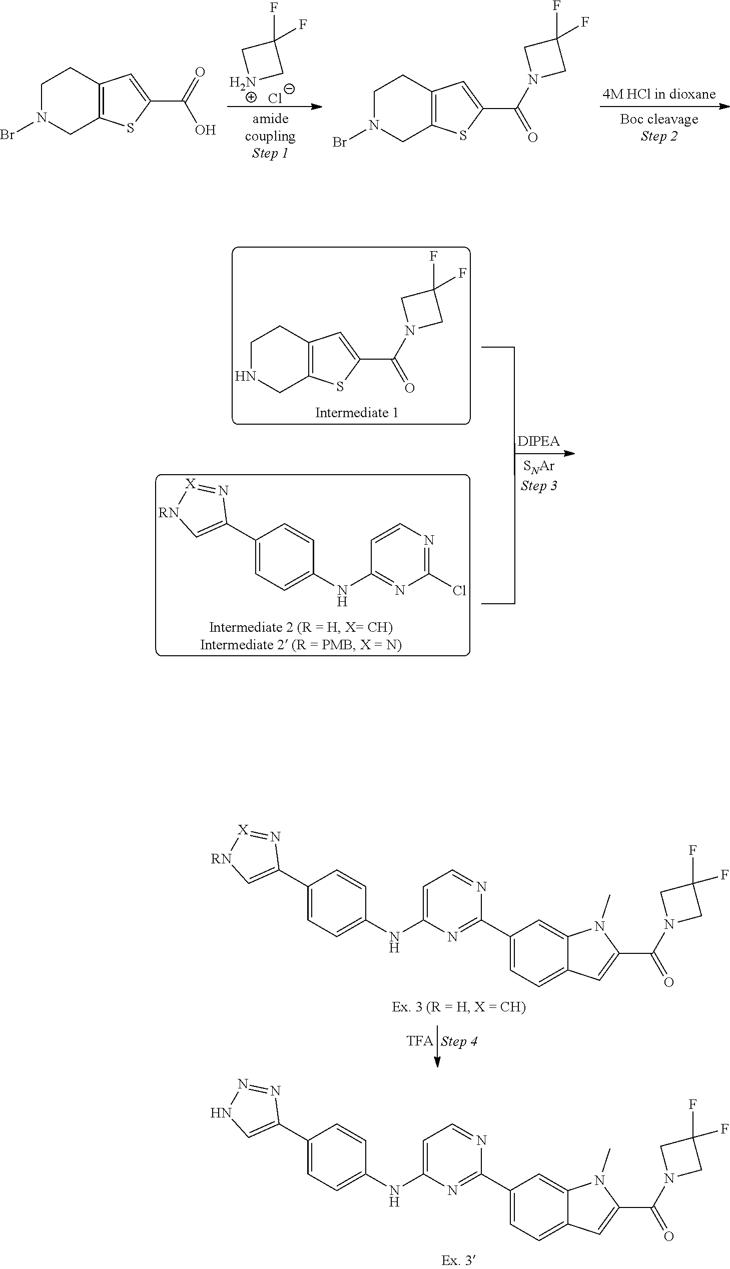

### Step 1 and Step 2: Synthesis of Intermediate 1: 3,3-difluoro-1-{4H,5H,6H,7H-thieno[2,3-c]pyridine-2-carbonyl}azetidine

6-[(tert-butoxy)carbonyl]-4H,5H,6H,7H-thieno[2,3-c]pyridine-2-carboxylic acid (0.20 g, 0.70 mmol, 1.00 equiv) was dissolved in DMF (5 mL), next to the reaction mixture were added DIPEA (0.50 mL, 2.83 mmol, 4.00 equiv) followed by HATU (0.32 g, 0.84 mmol, 1.20 equiv). The reaction mixture was stirred at room temperature for 15 min and 3,3-Difluoroazetidine hydrochloride (0.11 g, 0.85 mmol, 1.20 equiv) was added. The reaction mixture was allowed to stir at room temperature overnight. The reaction mixture was quenched by addition of water and the water layer was extracted with EtOAc. The combined organic layers were washed with brine, dried over Na₂SO₄, filtered and concentrated to give tert-butyl 2-(3,3-difluoroazetidine-1-carbonyl)-4H,5H,6H,7H-thieno[2,3-c]pyridine-6-carboxylate, [M+ACN]⁺=358.90. The crude material was used in the next step without further purification. To the obtained residue 2M HCl in dioxane (2.00 mL, 8.20 mmol, 11.00 equiv) was added and the mixture was stirred at room temperature for 12 h. The solvent was evaporated to dryness and the obtained HCl salt of 3,3-difluoro-1-{4H,5H,6H,7H-thieno[2,3-c]pyridine-2-carbonyl}azetidine (0.25 g), which was used directly for the next step. UPLC-MS, m/z: [M+ACN]⁺=299.80.

### Step 3: Synthesis of: 2-[2-(3,3-difluoroazetidine-1-carbonyl)-4H,5H,6H,7H-thieno[2,3-c]pyridin-6-yl]-N-[4-(1H-imidazol-5-yl)phenyl]pyrimidin-4-amine

To a solution of Intermediate 1 (0.17 g, 0.33 mmol, 1.00 equiv), in Acetonitrile (5.0 mL) was added N,N-Diisopropylethylamine (DIPEA) (0.23 mL, 1.33 mmol, 4.00 equiv) and the reaction mixture was stirred for 15 min at room temperature. Then Intermediate 2 (0.10 g, 0.33 mmol, 1.00 equiv) was added and the reaction mixture was allowed to stir at 95° C. overnight. Low conversion of starting materials was observed (acc. to UPLC-MS analysis). To the reaction mixture Cs₂CO₃ (0.21 g, 0.66 mmol, 2.00 equiv) was added and stirring was continued at the same temperature for additional 12 hr. Then the reaction mixture was quenched by addition of water and extracted with EtOAc. The combined organic layers were dried over Na₂SO₄, filtrated, concentrated and purified by preparative HPLC (Mobile phases: ACN+0.1% FA, H2O+0.1% FA Column C18 prep) to provide 2-[2-(3,3-difluoroazetidine-1-carbonyl)-4H,5H,6H,7H-thieno[2,3-c]pyridin-6-yl]-N-[4-(1H-imidazol-5-yl)phenyl]pyrimidin-4-amine (0.003 g, 2%). ¹H NMR (400 MHz, DMSO-d6) δ 9.37 (s, 1H), 8.42 (s, 5H), 7.97 (d, J=5.7 Hz, 1H), 7.67 (dd, J=26.6, 10.4 Hz, 6H), 7.51 (s, 1H), 7.34 (s, 1H), 6.11 (d, J=5.7 Hz, 1H), 4.98 (s, 2H), 4.04 (t, J=5.8 Hz, 3H), 2.75 (s, 3H). LCMS, m/z: [M+H]⁺=493.85.

### Step 4: 2-[2-(3,3-difluoroazetidine-1-carbonyl)-4H,5H,6H,7H-thieno[2,3-c]pyridin-6-yl]-N-[4-(1H-1,2,3-triazol-4-yl)phenyl]pyrimidin-4-amine

The 2-[2-(3,3-difluoroazetidine-1-carbonyl)-4H,5H,6H,7H-thieno[2,3-c]pyridin-6-yl]-N-(4-{1-[(4-methoxyphenyl)methyl]-1H-1,2,3-triazol-4-yl}phenyl)pyrimidin-4-amine (0.20 g, 0.20 mmol, 1.00 equiv) was dissolved in Trifluoroacetic acid (1.06 mL, 13.85 mmol, 70.00 equiv) and the reaction mixture was stirred at 100° C. overnight. After that time no partial conversion was observed. The reaction mixture was evaporated to dryness and purified using prep HPLC (Mobile phases: ACN+0.1% FA, H2O+0.1% FA, Column C18 prep) to give 22-[2-(3,3-difluoroazetidine-1-carbonyl)-4H,5H,6H,7H-thieno[2,3-c]pyridin-6-yl]-N-[4-(1H-1,2,3-triazol-4-yl)phenyl]pyrimidin-4-amine (0.025 g, 26%). ¹H NMR (400 MHz, DMSO-d6) δ 9.50 (s, 1H), 8.35 (s, 1H), 8.27 (s, 1H), 8.00 (d, J=5.7 Hz, 1H), 7.83 (d, J=8.7 Hz, 2H), 7.75 (d, J=8.7 Hz, 2H), 7.34 (s, 1H), 6.13 (d, J=5.7 Hz, 1H), 4.99 (s, 2H), 4.67 (s, 4H), 4.05 (t, J=5.7 Hz, 2H), 2.76 (d, J=6.0 Hz, 2H). Formic acid salt. LCMS, m/z: [M+H]+=495.11.

### Example 4

### Method A

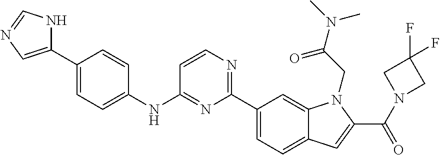 [SMILES: CN(C)C(=O)Cn1c(C(=O)N2CC(F)(F)C2)cc2ccc(-c3nccc(Nc4ccc(-c5cnc[nH]5)cc4)n3)cc21]

¹H NMR (300 MHz, DMSO-d6) δ 12.51 (s, 1H), 9.71 (s, 1H), 8.47 (s, 1H), 8.40 (d, J=5.9 Hz, 1H), 8.20 (dd, J=8.4, 1.3 Hz, 1H), 8.14 (s, 1H), 7.81 (d, J=2.6 Hz, 5H), 7.75 (d, J=8.4 Hz, 1H), 7.55 (s, 1H), 7.09 (s, 1H), 6.71 (d, J=5.9 Hz, 1H), 5.52 (s, 2H), 4.69 (d, J=89.6 Hz, 4H), 3.18 (s, 3H), 2.86 (s, 3H). Formic acid salt. LCMS, m/z: [M+H]⁺=557.33.

### Example 5

### Method A

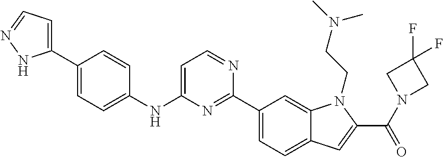 [SMILES: CN(C)CCn1c(C(=O)N2CC(F)(F)C2)cc2ccc(-c3nccc(Nc4ccc(-c5ccn[nH]5)cc4)n3)cc21]

¹H NMR (400 MHz, DMSO-d6) δ 9.66 (s, 1H), 8.55 (s, 1H), 8.40 (d, J=5.8 Hz, 1H), 8.19 (dd, J=8.4, 1.4 Hz, 1H), 8.16 (s, 1H), 7.80 (s, 4H), 7.73 (d, J=8.4 Hz, 1H), 7.70 (d, J=1.1 Hz, 1H), 7.51 (s, 1H), 7.00 (s, 1H), 6.71 (d, J=5.9 Hz, 1H), 4.87-4.37 (m, 6H), 2.14 (s, 6H). Two aliphatic protons CH₂ overlapped with H₂O. Formic acid salt. LCMS, m/z: [M+H]⁺=543.23.

### Example 6

### Method A

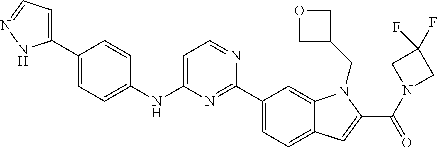 [SMILES: O=C(c1cc2ccc(-c3nccc(Nc4ccc(-c5ccn[nH]5)cc4)n3)cc2n1CC1COC1)N1CC(F)(F)C1]

¹H NMR (400 MHz, DMSO-d6) δ 9.70 (s, 1H), 8.64 (s, 1H), 8.41 (d, J=5.8 Hz, 1H), 8.27 (s, 1H), 8.21 (dd, J=8.4, 1.3 Hz, 1H), 7.82 (d, J=4.9 Hz, 4H), 7.75 (d, J=8.4 Hz, 1H), 7.70 (d, J=1.0 Hz, 1H), 7.49 (s, 1H), 7.12 (s, 1H), 6.73 (d, J=5.8 Hz, 1H), 5.02-4.41 (m, 10H), 3.62-3.52 (m, 1H). Formic acid salt. LCMS, m/z: [M+H]⁺=542.14.

### Example 7

### Method A

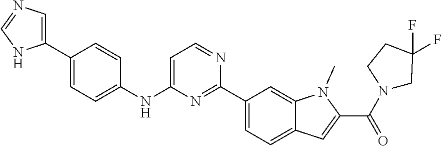 [SMILES: Cn1c(C(=O)N2CCC(F)(F)C2)cc2ccc(-c3nccc(Nc4ccc(-c5cnc[nH]5)cc4)n3)cc21]

¹H NMR (300 MHz, DMSO-d6) δ 9.68 (s, 1H), 8.54 (s, 1H), 8.40 (d, J=5.9 Hz, 1H), 8.24-8.14 (m, 2H), 7.87-7.77 (m, 4H), 7.74 (d, J=8.4 Hz, 1H), 7.70 (s, 1H), 7.53 (s, 1H), 7.02 (s, 1H), 6.72 (d, J=5.9 Hz, 1H), 4.31-3.96 (m, 4H), 3.95 (s, 3H), 3.91-3.71 (m, 2H). Formic acid salt. LCMS, m/z: [M+H]⁺=500.39.

### Example 8

### Method A

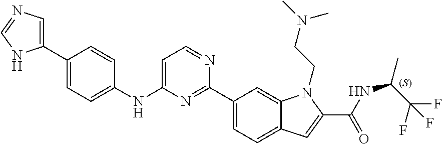

¹H NMR (400 MHz, DMSO-d6) δ 9.73 (s, 1H), 9.10 (d, J=8.8 Hz, 1H), 8.56 (s, 1H), 8.41 (d, J=5.8 Hz, 1H), 8.33 (s, 4H), 8.20 (dd, J=8.4, 1.3 Hz, 1H), 7.87-7.75 (m, 5H), 7.71 (d, J=1.0 Hz, 1H), 7.50 (s, 1H), 7.23 (s, 1H), 6.73 (d, J=5.8 Hz, 1H), 4.87 (dt, J=15.3, 7.7 Hz, 1H), 4.72 (t, J=6.9 Hz, 2H), 2.60 (t, J=6.8 Hz, 2H), 2.16 (s, 6H), 1.40 (d, J=7.0 Hz, 3H). Formic acid salt (1:4). LCMS, m/z: [M+H]⁺=563.23.

### Example 9

### Method A

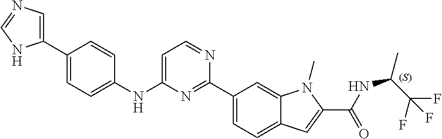

¹H NMR (300 MHz, DMSO-d6) δ 9.70 (s, 1H), 8.99 (d, J=8.9 Hz, 1H), 8.56 (s, 1H), 8.41 (d, J=5.8 Hz, 1H), 8.31 (s, 2H), 8.20 (dd, J=8.4, 1.3 Hz, 1H), 7.86-7.75 (m, 4H), 7.70 (d, J=1.1 Hz, 1H), 7.53 (s, 1H), 7.30 (s, 1H), 6.73 (d, J=5.9 Hz, 1H), 4.99-4.80 (m, 1H), 4.09 (s, 3H), 1.40 (d, J=7.1 Hz, 3H). Formic acid salt. LCMS, m/z: [M+H]⁺=506.18.

### Example 10

### Method A

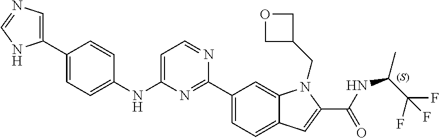

¹H NMR (400 MHz, DMSO-d6) δ 12.12 (s, 1H), 9.70 (s, 1H), 9.06 (d, J=8.9 Hz, 1H), 8.63 (s, 1H), 8.42 (d, J=5.8 Hz, 1H), 8.21 (d, J=8.7 Hz, 1H), 7.88-7.76 (m, 5H), 7.71 (s, 1H), 7.52 (s, 1H), 7.32 (s, 1H), 6.73 (d, J=5.9 Hz, 1H), 5.00 (dt, J=15.4, 7.5 Hz, 2H), 4.95-4.84 (m, 1H), 4.61 (dt, J=7.9, 6.1 Hz, 2H), 4.46 (t, J=6.1 Hz, 2H), 3.51 (p, J=7.0 Hz, 1H), 1.40 (d, J=7.0 Hz, 3H). LCMS, m/z: [M+H]⁺=562.51.

### Example 11

### Method A

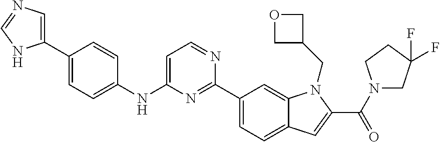 [SMILES: O=C(c1cc2ccc(-c3nccc(Nc4ccc(-c5cnc[nH]5)cc4)n3)cc2n1CC1COC1)N1CCC(F)(F)C1]

¹H NMR (400 MHz, DMSO-d6) δ 12.11 (s, 1H), 9.69 (s, 1H), 8.61 (s, 1H), 8.41 (d, J=5.8 Hz, 1H), 8.20 (dd, J=8.4, 1.3 Hz, 1H), 7.82 (s, 4H), 7.74 (d, J=8.5 Hz, 1H), 7.71 (d, J=1.1 Hz, 1H), 7.52 (s, 1H), 7.04 (s, 1H), 6.72 (d, J=5.8 Hz, 1H), 4.81 (d, J=7.4 Hz, 2H), 4.62 (dd, J=7.8, 6.1 Hz, 2H), 4.42 (s, 2H), 3.98 (dd, J=84.9, 48.3 Hz, 4H), 3.47 (s, 2H). One proton CH overlapped with H₂O. LCMS, m/z: [M+H]⁺=556.14.

### Example 12

### Method A

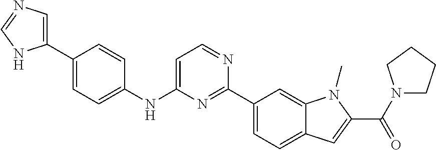 [SMILES: Cn1c(C(=O)N2CCCC2)cc2ccc(-c3nccc(Nc4ccc(-c5cnc[nH]5)cc4)n3)cc21]

¹H NMR (400 MHz, DMSO-d6) δ 9.68 (s, 1H), 8.53 (s, 1H), 8.40 (d, J=5.8 Hz, 1H), 8.30 (s, 3H), 8.18 (dd, J=8.4, 1.4 Hz, 1H), 7.84-7.77 (m, 4H), 7.72 (d, J=8.4 Hz, 1H), 7.70 (d, J=1.1 Hz, 1H), 7.52 (s, 1H), 6.90 (s, 1H), 6.72 (d, J=5.9 Hz, 1H), 3.93 (s, 3H), 3.66 (t, J=6.3 Hz, 2H), 3.55 (t, J=6.6 Hz, 2H), 1.91 (d, J=8.3 Hz, 4H). Formic acid salt (1:3). LCMS, m/z: [M+H]⁺=464.39.

### Example 13

### Method A

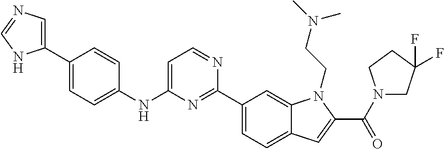 [SMILES: CN(C)CCn1c(C(=O)N2CCC(F)(F)C2)cc2ccc(-c3nccc(Nc4ccc(-c5cnc[nH]5)cc4)n3)cc21]

¹H NMR (400 MHz, DMSO-d₆) δ 9.70 (s, 1H), 8.57 (s, 1H), 8.42 (d, J=5.8 Hz, 1H), 8.21 (dd, J=8.5, 1.4 Hz, 1H), 8.14 (s, 1H), 7.87-7.73 (m, 5H), 7.55 (s, 1H), 7.06 (s, 1H), 6.73 (d, J=5.9 Hz, 1H), 4.66 (s, 2H), 4.20 (s, 2H), 4.00 (s, 2H), 3.80 (s, 2H), 3.05 (s, 2H). Six aliphatic protons 2×CH₃ overlapped with DMSO-d6. LCMS, m/z: [M+H]⁺=557.20.

### Example 14

### Method A

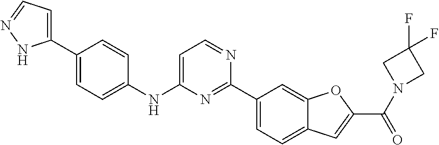 [SMILES: O=C(c1cc2ccc(-c3nccc(Nc4ccc(-c5ccn[nH]5)cc4)n3)cc2o1)N1CC(F)(F)C1]

¹H NMR (400 MHz, DMSO-d6) δ 9.75 (s, 1H), 8.58 (s, 1H), 8.44-8.39 (m, 2H), 8.28 (s, 2H), 7.91 (d, J=8.3 Hz, 1H), 7.80 (q, J=8.6 Hz, 4H), 7.70 (d, J=1.1 Hz, 1H), 7.66 (d, J=1.0 Hz, 1H), 7.55 (s, 1H), 6.77 (d, J=5.9 Hz, 1H), 5.12 (s, 2H), 4.56 (s, 2H). Formic acid salt (1:2). LCMS, m/z: [M+H]⁺=473.29.

### Example 15

### Method B

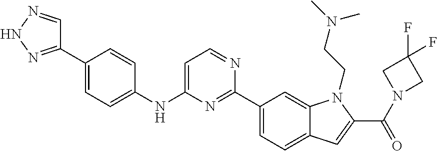 [SMILES: CN(C)CCn1c(C(=O)N2CC(F)(F)C2)cc2ccc(-c3nccc(Nc4ccc(-c5cn[nH]n5)cc4)n3)cc21]

¹H NMR (300 MHz, DMSO-d6) δ 9.82 (s, 1H), 8.56 (s, 1H), 8.44 (d, J=5.8 Hz, 1H), 8.28 (s, 1H), 8.20 (dd, J=8.4, 1.3 Hz, 1H), 8.14 (s, 1H), 7.97-7.87 (m, 4H), 7.74 (d, J=8.4 Hz, 1H), 7.01 (s, 1H), 6.75 (d, J=5.8 Hz, 1H), 4.64 (t, J=6.0 Hz, 6H), 2.17 (s, 6H). Two aliphatic protons CH₂ overlapped with DMSO. Formic acid salt. LCMS, m/z: [M+H]⁺=544.38.

### Example 16

### Method B

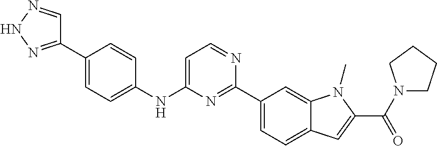 [SMILES: Cn1c(C(=O)N2CCCC2)cc2ccc(-c3nccc(Nc4ccc(-c5cn[nH]n5)cc4)n3)cc21]

¹H NMR (400 MHz, DMSO-d6) δ 9.84 (s, 1H), 8.53 (s, 1H), 8.44 (s, 1H), 8.40 (s, 3H), 8.30 (s, 1H), 8.19 (dd, J=8.4, 1.4 Hz, 1H), 7.93 (q, J=8.7 Hz, 4H), 7.73 (d, J=8.4 Hz, 1H), 6.91 (s, 1H), 6.76 (d, J=5.8 Hz, 1H), 3.94 (s, 3H), 3.67 (s, 2H), 3.54 (d, J=6.9 Hz, 2H), 1.90 (s, 4H). Formic acid salt (1:3). LCMS, m/z: [M+H]⁺=465.37.

### Example 17

### Method B

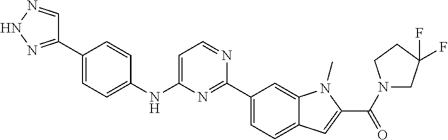 [SMILES: Cn1c(C(=O)N2CCC(F)(F)C2)cc2ccc(-c3nccc(Nc4ccc(-c5cn[nH]n5)cc4)n3)cc21]

¹H NMR (400 MHz, DMSO-d6) δ 9.84 (s, 1H), 8.55 (s, 1H), 8.44 (d, J=5.8 Hz, 1H), 8.35 (s, 1H), 8.30 (s, 1H), 8.20 (dd, J=8.4, 1.4 Hz, 1H), 7.93 (q, J=8.8 Hz, 4H), 7.75 (d, J=8.5 Hz, 1H), 7.02 (s, 1H), 6.76 (d, J=5.8 Hz, 1H), 4.20 (s, 2H), 3.95 (s, 4H), 3.81 (s, 1H). Two aliphatic protons CH₂ overlapped with H₂O. Formic acid salt. LCMS, m/z: [M+H]⁺=501.20.

### Example 18

### Method B

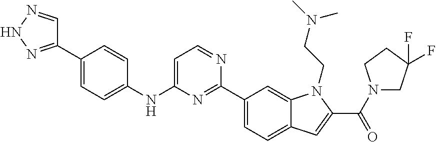 [SMILES: CN(C)CCn1c(C(=O)N2CCC(F)(F)C2)cc2ccc(-c3nccc(Nc4ccc(-c5cn[nH]n5)cc4)n3)cc21]

¹H NMR (400 MHz, DMSO-d₆) δ 9.81 (s, 1H), 8.55 (s, 1H), 8.44 (d, J=5.8 Hz, 1H), 8.26 (s, 1H), 8.19 (dd, J=8.4, 1.3 Hz, 1H), 8.14 (s, 1H), 7.92 (q, J=8.7 Hz, 4H), 7.74 (d, J=8.4 Hz, 1H), 6.97 (d, J=3.2 Hz, 1H), 6.75 (d, J=5.8 Hz, 1H), 4.57 (s, 2H), 4.14 (s, 2H), 3.96 (s, 2H), 3.78 (s, 2H), 2.19 (s, 6H). Two aliphatic protons CH₂ overlapped with DMSO-d6. Formic acid salt. LCMS, m/z: [M+H]⁺=558.16.

### Example 19

### Method B

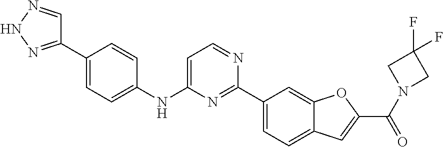 [SMILES: O=C(c1cc2ccc(-c3nccc(Nc4ccc(-c5cn[nH]n5)cc4)n3)cc2o1)N1CC(F)(F)C1]

¹H NMR (400 MHz, DMSO-d6) δ 9.91 (s, 1H), 8.58 (s, 1H), 8.46 (d, J=5.8 Hz, 1H), 8.42 (dd, J=8.2, 1.4 Hz, 1H), 8.39 (s, 1H), 8.32 (s, 1H), 7.96-7.88 (m, 5H), 7.67 (d, J=1.0 Hz, 1H), 6.81 (d, J=5.9 Hz, 1H), 5.12 (s, 2H), 4.56 (s, 2H). Formic acid salt. LCMS, m/z: [M+H]⁺=474.20.

### Example 20

### Method B

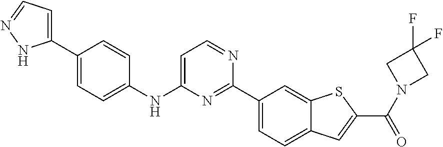 [SMILES: O=C(c1cc2ccc(-c3nccc(Nc4ccc(-c5ccn[nH]5)cc4)n3)cc2s1)N1CC(F)(F)C1]

¹H NMR (400 MHz, DMSO-d6) δ 12.14 (s, 1H), 9.74 (s, 1H), 8.98 (d, J=1.4 Hz, 1H), 8.46 (dd, J=8.5, 1.5 Hz, 1H), 8.43 (d, J=5.9 Hz, 1H), 8.08 (d, J=8.5 Hz, 1H), 8.01 (s, 1H), 7.79 (s, 4H), 7.71 (d, J=1.1 Hz, 1H), 7.59 (d, J=20.2 Hz, 1H), 6.77 (d, J=5.9 Hz, 1H), 4.85 (d, J=199.8 Hz, 4H). Contains residual of Formic acid. LCMS, m/z: [M+H]⁺=489.10.

### Example 21

### Method B

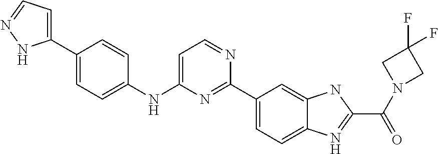 [SMILES: O=C(c1nc2ccc(-c3nccc(Nc4ccc(-c5ccn[nH]5)cc4)n3)cc2[nH]1)N1CC(F)(F)C1]

¹H NMR (400 MHz, DMSO-d6) δ 13.60 (d, J=36.6 Hz, 1H), 9.72 (s, 1H), 8.68 (d, J=34.4 Hz, 1H), 8.50-8.33 (m, 2H), 8.20 (d, J=3.9 Hz, 2H), 7.81 (s, 5H), 7.71 (d, J=1.1 Hz, 1H), 7.53 (s, 1H), 6.73 (d, J=5.8 Hz, 1H), 5.16 (d, J=13.0 Hz, 2H), 4.62 (t, J=12.3 Hz, 2H). Formic acid salt. LCMS, m/z: [M+H]⁺=472.88.

### Example 22

### Method B

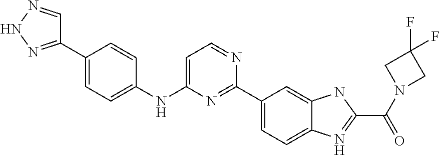 [SMILES: O=C(c1nc2ccc(-c3nccc(Nc4ccc(-c5cn[nH]n5)cc4)n3)cc2[nH]1)N1CC(F)(F)C1]

¹H NMR (400 MHz, DMSO-d6) δ 13.65 (s, 1H), 9.85 (s, 1H), 8.67 (s, 1H), 8.44 (d, J=5.8 Hz, 1H), 8.37 (s, 2H), 8.30 (s, 1H), 7.93 (s, 4H), 7.81 (d, J=12.2 Hz, 1H), 6.77 (d, J=5.8 Hz, 1H), 5.18 (t, J=12.3 Hz, 2H), 4.62 (t, J=12.2 Hz, 2H). LCMS, m/z: [M+H]⁺=474.25.

### Example 23

### Method C

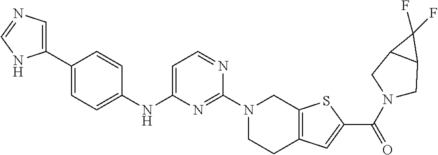 [SMILES: O=C(c1cc2c(s1)CN(c1nccc(Nc3ccc(-c4cnc[nH]4)cc3)n1)CC2)N1CC2C(C1)C2(F)F]

¹H NMR (400 MHz, DMSO-d₆) δ 9.34 (s, 1H), 8.17 (s, 2H), 7.97 (d, J=5.7 Hz, 1H), 7.76-7.67 (m, 3H), 7.63 (d, J=8.4 Hz, 2H), 7.50 (s, 1H), 7.38 (s, 1H), 6.10 (d, J=5.7 Hz, 1H), 4.96 (s, 2H), 4.12 (s, 1H), 4.04 (t, J=5.8 Hz, 4H), 3.77 (s, 2H), 2.75 (s, 2H). Formic acid salt (1:2). LCMS, m/z: [M+H]⁺=520.11.

### Example 24

### Method C

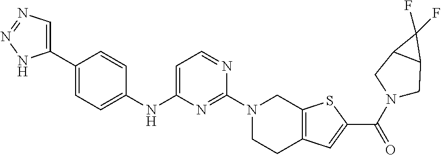 [SMILES: O=C(c1cc2c(s1)CN(c1nccc(Nc3ccc(-c4cnn[nH]4)cc3)n1)CC2)N1CC2C(C1)C2(F)F]

¹H NMR (400 MHz, DMSO-d6) δ 9.49 (s, 1H), 8.32 (s, 2H), 8.27 (s, 1H), 8.00 (d, J=5.7 Hz, 1H), 7.88-7.80 (m, 2H), 7.75 (d, J=8.7 Hz, 2H), 7.38 (s, 1H), 6.13 (d, J=5.7 Hz, 1H), 4.97 (s, 2H), 4.12 (s, 1H), 4.05 (t, J=5.9 Hz, 4H), 3.77 (s, 2H), 2.76 (s, 3H), 2.61 (s, 1H). Formic acid salt (1:2). LCMS, m/z: [M+H]⁺=521.09.

### Example 25

### Method C

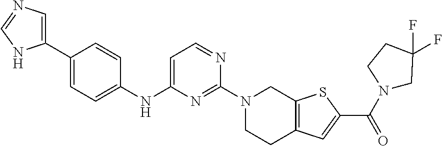 [SMILES: O=C(c1cc2c(s1)CN(c1nccc(Nc3ccc(-c4cnc[nH]4)cc3)n1)CC2)N1CCC(F)(F)C1]

¹H NMR (400 MHz, DMSO-d6) δ 12.12 (s, 1H), i9.34 (s, 1H), 8.28 (s, 2H), 7.97 (d, J=5.6 Hz, 1H), 7.73 (s, 2H), 7.68 (d, J=1.1 Hz, 1H), 7.63 (d, J=7.9 Hz, 2H), 7.54 (s, 1H), 7.42 (s, 1H), 6.10 (d, J=5.7 Hz, 1H), 4.97 (s, 2H), 4.21 (bs, 1H), 4.04 (t, J=5.8 Hz, 3H), 3.96-3.69 (m, 4H), 2.76 (s, 2H). Formic acid salt (1:2). LCMS, m/z: [M+H]⁺=508.42.

### Comparative Example

The structure of comparative Compound A is provided below:

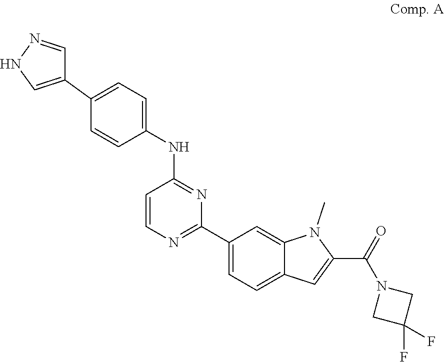 [SMILES: Cn1c(C(=O)N2CC(F)(F)C2)cc2ccc(-c3nccc(Nc4ccc(-c5cn[nH]c5)cc4)n3)cc21]

which may be prepared according to the methods found in WO 2019/001572.

### Example 26

ADP-Glo kinase assay (Promega). The ADP-Glo™ Kinase Assay is a luminescent ADP detection assay that allows measurement of kinase activity based on the amount of ADP produced during a kinase reaction. The kinase reaction is performed in the presence of ATP, S6K substrate (KRRRLASLR) and the ROCK kinase in the appropriate Kinase Reaction Buffer (1×). Upon termination of the reaction, the unconsumed ATP is depleted by addition of ADP-Glo™ Reagent. Next, Kinase Detection Reagent is added to convert ADP to ATP and allow the newly synthesized ATP to be measured using a luciferase/luciferin reaction. The luminescence is proportional to the produced ADP and consequently to the kinase activity. The ADP-Glo™ Kinase Assay was performed in a 384 well plate format as follows.

Kinase Detection Reagent Preparation: The Kinase Detection Buffer was thawed at room temperature (RT). If a precipitate was present, the buffer was incubated at 37° C. with constant swirling for 15 minutes to dissolve the precipitate or the precipitate was removed from the buffer by carefully pipetting the supernatant from the bottle. After equilibrating both the Kinase Detection Buffer and Kinase Detection Substrate to room temperature, the entire volume of Kinase Detection Buffer was transferred into the bottle containing Kinase Detection Substrate to reconstitute the lyophilized substrate, followed by gentile mixing to obtain a homogeneous solution.

Kinase Reaction Buffer Preparation: Kinase reaction buffer was prepared freshly before each experiment. The formulation of this buffer is provided in Table 1 below:

TABLE 1
Formulation of kinase reaction buffer
(1x) for ADP-Glo kinase assay.

| | | |
|---|---|---|
| Reagent: | Final concentration | Stock concentration |

| | | | |
|---|---|---|---|
| Tris, pH 7.5 | 40 | mM | 500 mM in H₂O |
| DTT | 0.05 | mM | 100 mM in H₂O |
| MgCl₂ | 20 | mM | 500 mM in H₂O |
| BSA | 0.1 | mg/mL | 5.0 mg/mL |

ADP-Glo Reagents Preparation. Three mixes were prepared on ice before running the assay:
Mix 1—containing ROCK1 or ROCK2 in Kinase Reaction Buffer (1×)
Mix 2—containing S6K peptide in Kinase Reaction Buffer (1×)
Mix 3—containing ATP in Kinase Reaction Buffer (1×)

The final conditions for performing the ADP-Glo kinase assay are proved in Table 2 below:

TABLE 2
Final conditions for ADP-Glo kinase assay for
ROCK (concentration in final volume 5 μL).

| | | |
|---|---|---|
| Final concentrations: | ROCK1 | ROCK2 |

| | | | | |
|---|---|---|---|---|
| Kinase | 0.1 | ng/μL | 0.1 | ng/μL |
| S6K substrate | 170 | μM | 170 | μM |
| ATP | 10 | μM | 40 | μM |
| Timepoint | 120 | min | 120 | min |

| | | |
|---|---|---|
| DMSO | 1% | 1% |

The ratios of reagent volumes in the assay were 1:1:2 of kinase reaction to ADP-Glo™ Reagent to Kinase Detection Reagent. For 384-well plates, volumes were: 5 μl of kinase reaction, 5 μl of ADP-Glo™ Reagent and 10 μl of Kinase Detection Reagent.

Generating a standard curve for conversion of ATP to ADP. To estimate the amount of ADP produced in the kinase reaction, a standard curve that represents the luminescence corresponding to the conversion of ATP to ADP was prepared based on the ATP concentration used in the kinase reaction. These conversion curves represent the amounts of ATP and ADP available in a reaction at the specified conversion percentage ranging from 100% conversion to 0% conversion. The standard samples used to generate an ATP-to-ADP conversion curve are created by combining the appropriate volumes of ATP and ADP stock solutions.

### Determination of IC₅₀ Values of Kinase Inhibitors

Each plate contained a positive control (1 μM Comp. A for ROCK2 and 1 μM RKI-1447 for ROCK1); a vehicle control (1% DMSO); a blank control (kinase reaction buffer (1×)); a low control (substrate and ATP, without kinase); and an autophosphorylation control (kinase and ATP, without substrate). Test and reference compounds were diluted in 100% DMSO to obtain 10 mM stock solutions. Each compound was tested in 8-serial dilutions in duplicate. The final concentration of DMSO in the reaction was 1% (up to 50 nL).

5 μl/well Kinase Reaction Buffer (1×) was dispensed to blank control wells. 2 μl/well Kinase Reaction Buffer (1×) was dispensed to control without protein (low control) and without substrate (autophosphorylation) wells. The plate was sealed with adhesive film followed by a short spin (1 min, 1000 rpm).

2 μl/well kinase (2.5×) solution was dispensed into relevant wells of the assay plate. The plate was sealed with adhesive film followed by a short spin (1 min, 1000 rpm).

2 μl/well S6K substrate (2.5×) solution was dispensed into relevant wells of the assay plate. The plate was sealed with adhesive film followed by a short spin (1 min, 1000 rpm).

1 μl/well ATP (5×) solution was dispensed into relevant wells of the assay plate. The final volume of reaction was 15 μl. The plate was sealed with adhesive film followed by a short spin (1 min, 1000 rpm).

The plate was incubated at 25° C. for 120 minutes with 450 rpm shaking speed.

After the incubation was completed the kinase reaction was terminated, residual ATP was depleted from the kinase reaction and ADP was converted to ATP, that is measured in luciferase/luciferin reaction. 5 μl of ADP-Glo™ Reagent was dispensed to stop the kinase reaction and deplete the unconsumed ATP. The plate was sealed with adhesive film followed by a short spin (1 min, 1000 rpm), and was incubated at 25° C. for 40 minutes with 450 rpm shaking speed.

10 μl of Kinase Detection Reagent was dispensed to convert ADP to ATP and introduce luciferase and luciferin to detect ATP. The plate was sealed with adhesive film followed by a short spin (1 min, 1000 rpm), and was incubated at 25° C. for 60 minutes with 450 rpm shaking speed.

Following a 5-minute dark adaptation, luminescence was measured using a PHERAstar FSX Multimode Plate Reader (BMG Labtech).

IC₅₀ parameters were calculated after normalization using GraphPad Prism 7.04 software, using 4-parameter model: log(inhibitor) vs. response—variable slope.

| Example # | ROCK2 | ROCK1 |
|---|---|---|
| Structure | IC50 (nM) | IC50 (nM) |
| Comp. A | 2.29 | 3212.91 |
| Example 1 | 473 | >50000 |
| 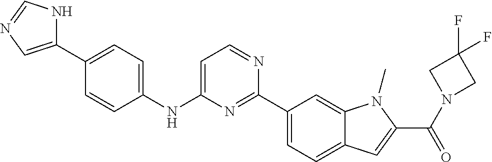 [SMILES: Cn1c(C(=O)N2CC(F)(F)C2)cc2ccc(-c3nccc(Nc4ccc(-c5cnc[nH]5)cc4)n3)cc21] | | |
| Example 2 | 36.67 | >50000 |
| 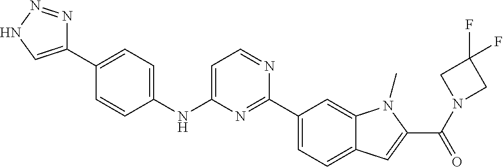 [SMILES: Cn1c(C(=O)N2CC(F)(F)C2)cc2ccc(-c3nccc(Nc4ccc(-c5c[nH]nn5)cc4)n3)cc21] | | |
| Example 4 | 1000 | >50000 |
| 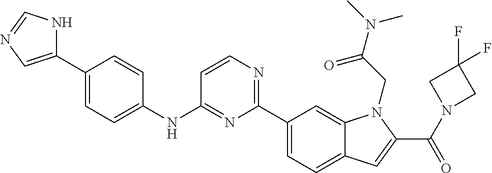 [SMILES: CN(C)C(=O)Cn1c(C(=O)N2CC(F)(F)C2)cc2ccc(-c3nccc(Nc4ccc(-c5cnc[nH]5)cc4)n3)cc21] | | |
| Example 5 | 10000 | >50000 |
| 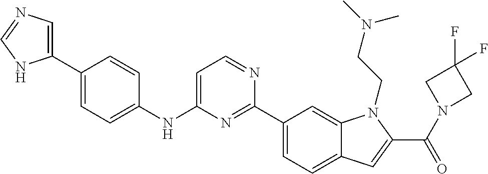 [SMILES: CN(C)CCn1c(C(=O)N2CC(F)(F)C2)cc2ccc(-c3nccc(Nc4ccc(-c5cnc[nH]5)cc4)n3)cc21] | | |
| Example 15 | 748.53 | 280.91 |
| 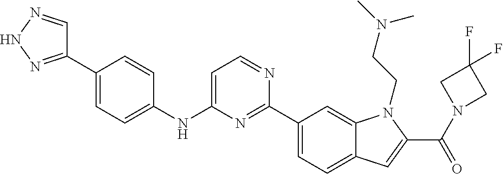 [SMILES: CN(C)CCn1c(C(=O)N2CC(F)(F)C2)cc2ccc(-c3nccc(Nc4ccc(-c5cn[nH]n5)cc4)n3)cc21] | | |
| Example 3′ | 961.37 | 2734.53 |
| 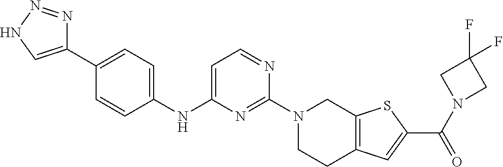 [SMILES: O=C(c1cc2c(s1)CN(c1nccc(Nc3ccc(-c4c[nH]nn4)cc3)n1)CC2)N1CC(F)(F)C1] | | |
| Example 6 | 958.42 | 1987.20 |
| 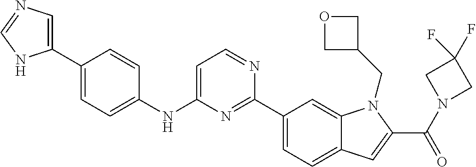 [SMILES: O=C(c1cc2ccc(-c3nccc(Nc4ccc(-c5cnc[nH]5)cc4)n3)cc2n1CC1COC1)N1CC(F)(F)C1] | | |
| Example 7 | 1371.63 | 2104.54 |
| 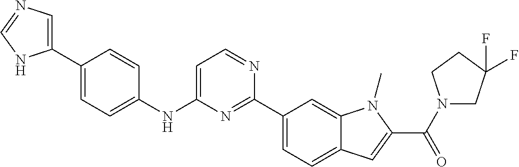 [SMILES: Cn1c(C(=O)N2CCC(F)(F)C2)cc2ccc(-c3nccc(Nc4ccc(-c5cnc[nH]5)cc4)n3)cc21] | | |
| Example 8 | 2761.14 | 10744.61 |
| 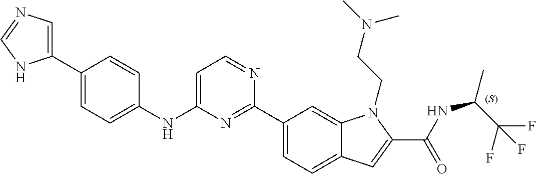 | | |
| Example 9 | 658.67 | 745.57 |
| 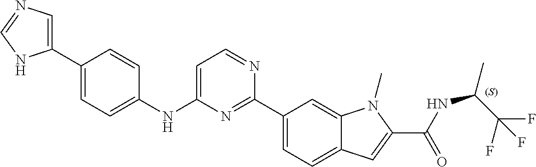 | | |
| Example 10 | 1810.41 | 4004.45 |
| 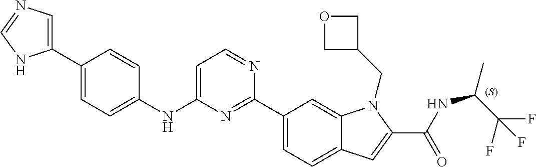 | | |
| Example 11 | 3574.74 | 3681.29 |
| 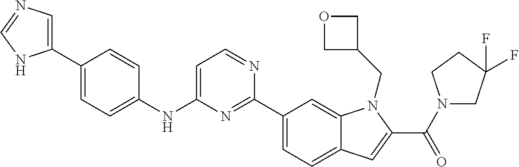 [SMILES: O=C(c1cc2ccc(-c3nccc(Nc4ccc(-c5cnc[nH]5)cc4)n3)cc2n1CC1COC1)N1CCC(F)(F)C1] | | |
| Example 12 | 10000 | 2279.88 |
| 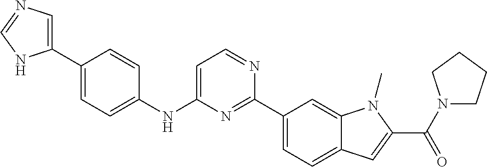 [SMILES: Cn1c(C(=O)N2CCCC2)cc2ccc(-c3nccc(Nc4ccc(-c5cnc[nH]5)cc4)n3)cc21] | | |
| Example 16 | 2085.65 | 792.36 |
| 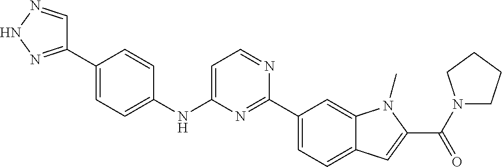 [SMILES: Cn1c(C(=O)N2CCCC2)cc2ccc(-c3nccc(Nc4ccc(-c5cn[nH]n5)cc4)n3)cc21] | | |
| Example 17 | 360.28 | 437.20 |
| 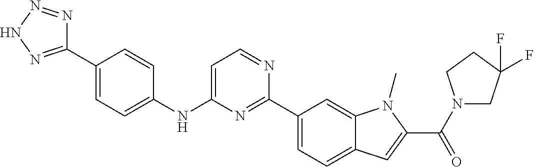 [SMILES: Cn1c(C(=O)N2CCC(F)(F)C2)cc2ccc(-c3nccc(Nc4ccc(-c5nn[nH]n5)cc4)n3)cc21] | | |
| Example 18 | 898.35 | 23249.09 |
| 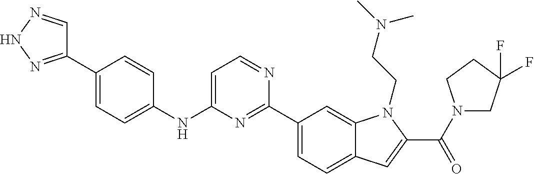 [SMILES: CN(C)CCn1c(C(=O)N2CCC(F)(F)C2)cc2ccc(-c3nccc(Nc4ccc(-c5cn[nH]n5)cc4)n3)cc21] | | |
| Example 13 | 10000 | 4293.96 |
| 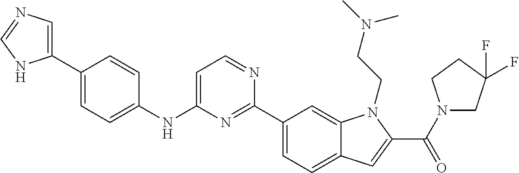 [SMILES: CN(C)CCn1c(C(=O)N2CCC(F)(F)C2)cc2ccc(-c3nccc(Nc4ccc(-c5cnc[nH]5)cc4)n3)cc21] | | |
| Example 14 | 10000 | 4707.06 |
| 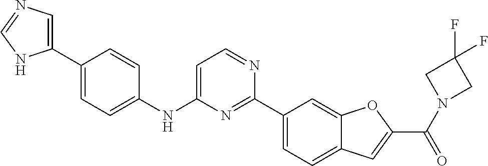 [SMILES: O=C(c1cc2ccc(-c3nccc(Nc4ccc(-c5cnc[nH]5)cc4)n3)cc2o1)N1CC(F)(F)C1] | | |
| Example 24 | 207.28 | 1217.76 |
| 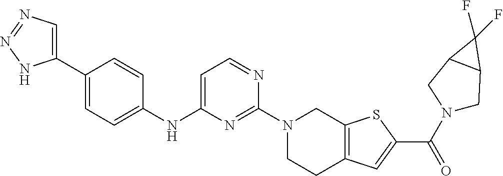 [SMILES: O=C(c1cc2c(s1)CN(c1nccc(Nc3ccc(-c4cnn[nH]4)cc3)n1)CC2)N1CC2C(C1)C2(F)F] | | |
| Example 19 | 10000 | 2008.99 |
| 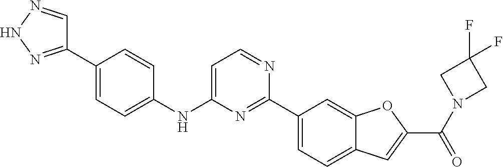 [SMILES: O=C(c1cc2ccc(-c3nccc(Nc4ccc(-c5cn[nH]n5)cc4)n3)cc2o1)N1CC(F)(F)C1] | | |
| Example 23 | 334.05 | 8909.87 |
| 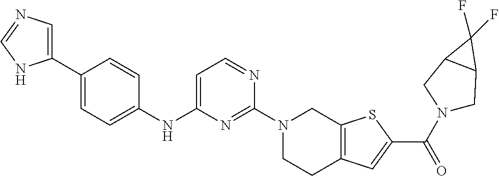 [SMILES: O=C(c1cc2c(s1)CN(c1nccc(Nc3ccc(-c4cnc[nH]4)cc3)n1)CC2)N1CC2C(C1)C2(F)F] | | |
| Example 20 | 6422.08 | 23349.74 |
| 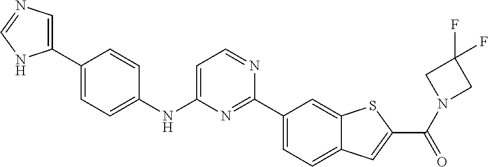 [SMILES: O=C(c1cc2ccc(-c3nccc(Nc4ccc(-c5cnc[nH]5)cc4)n3)cc2s1)N1CC(F)(F)C1] | | |
| Example 21 | 8233.95 | 7777.88 |
| 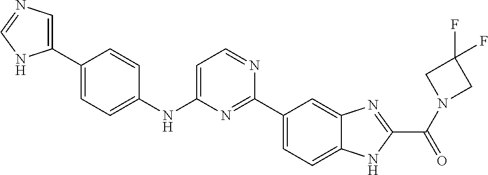 [SMILES: O=C(c1nc2cc(-c3nccc(Nc4ccc(-c5cnc[nH]5)cc4)n3)ccc2[nH]1)N1CC(F)(F)C1] | | |
| Example 22 | 827.03 | 25187.98 |
| 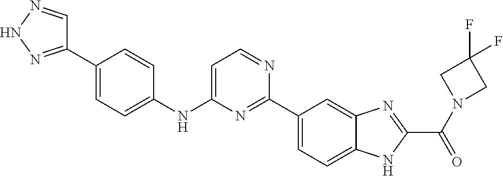 [SMILES: O=C(c1nc2cc(-c3nccc(Nc4ccc(-c5cn[nH]n5)cc4)n3)ccc2[nH]1)N1CC(F)(F)C1] | | |
| Example 25 | 2455.22 | 6846.37 |
| 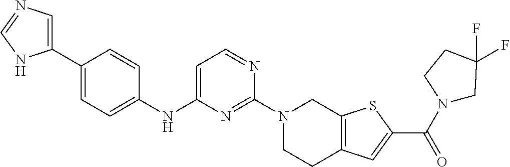 [SMILES: O=C(c1cc2c(s1)CN(c1nccc(Nc3ccc(-c4cnc[nH]4)cc3)n1)CC2)N1CCC(F)(F)C1] | | |

### Example 28

Selective ROCK2 Inhibition Down-Regulates IL-17 Secretion Via pSTAT3-Dependent Mechanism

Rho kinase (ROCK) are members of the serine/threonine kinase family, often studied for their role in cell morphology, motility, and shape through effects on the cytoskeleton. Although two isoforms of ROCK, ROCK1 and ROCK2 share more than 90% homology within their kinase domain, the function of these proteins is not redundant and depends on the cellular system where ROCKs are expressed and activated. Recent studies demonstrated that ROCK2, but not ROCK1 regulates a pro-inflammatory IL-17-producing lineage of T cells termed Th17 cells via STAT3-dependent mechanism. Dysregulated activation of Th17 cells and STAT3 phosphorylation have been implicated in the pathogenesis of plethora of inflammatory as well as fibrotic pathologies. Moreover, selective ROCK2 inhibition shifts the balance between pro-inflammatory Th17 and immune-suppressive regulatory T cells (Tregs) by increasing STAT5 phosphorylation and IL-10 secretion.

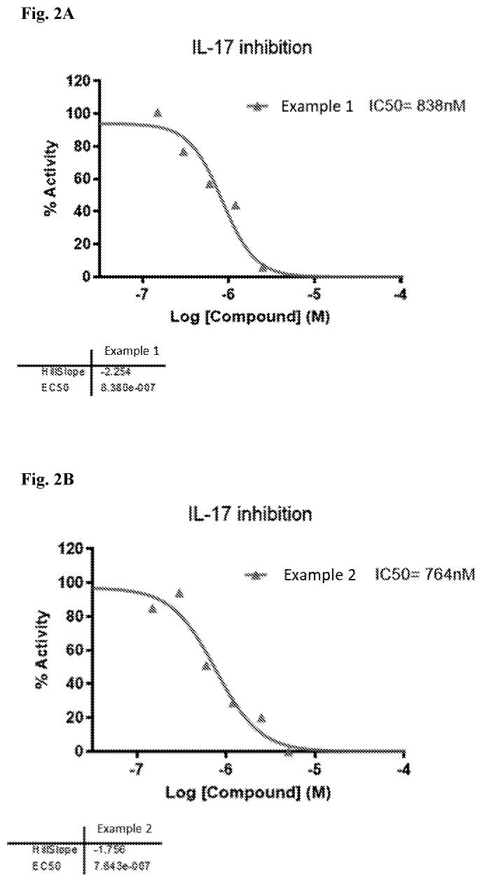

By using stimulatory antibodies against CD3 and CD28 (anti-CD3/CD28) in combination of IL-1β and TGF-β (Th17-skewing conditions), we found that novel selective ROCK2 inhibitors, such as the compounds of Example 1 and Example 2, robustly down-regulate IL-17 secretion in Th17-skewed human CD4+ T cells in a dose-dependent manner with EC50s at 838 nM and 764 nM, respectively (see FIGS. 2A and 2B). In this study, peripheral blood CD4⁺ T cells were treated with the indicated doses of the ROCK2 inhibitors and then stimulated by anti-CD3/28 mAbs, IL-1β (50 ng/mL) and TGF-β (5 ng/mL) for 48 hours. The supernatants were analyzed for IL-17 by ELISA. One representative of three different experiments is shown.

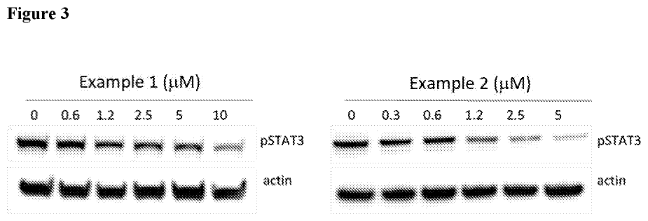

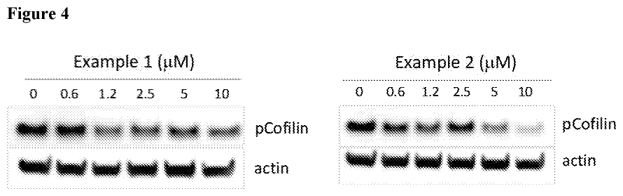

The induction and expression of IL-17 as well as other pro-inflammatory cytokines has been shown to be dependent on activation and phosphorylation of certain transcription factors including STAT3. Treatment of human CD4⁺ T cells with selective ROCK2 inhibitors during Th17-skewing conditions leads to a dose-dependent reduction of STAT3 phosphorylation (FIG. 3), consistent with the ability of the compounds of Example 1 and Example 2 to down-regulates IL-17 secretion as well as pCofilin (a classical down-stream ROCK target) under the same stimulatory conditions (FIG. 4). In these studies, peripheral blood CD4⁺ T cells were treated with indicated doses of inhibitors and then stimulated by anti-CD3/28 mAbs, IL-1β (50 ng/mL) and TGF-β (5 ng/mL) for 2 hours. The cell lysates were prepared and analyzed by Western Blot. One representative of three different experiments is shown.

### Example 27

### Selective ROCK2 Inhibitors Down-Regulate Pro-Fibrogenic Gene Expression in Fibroblasts

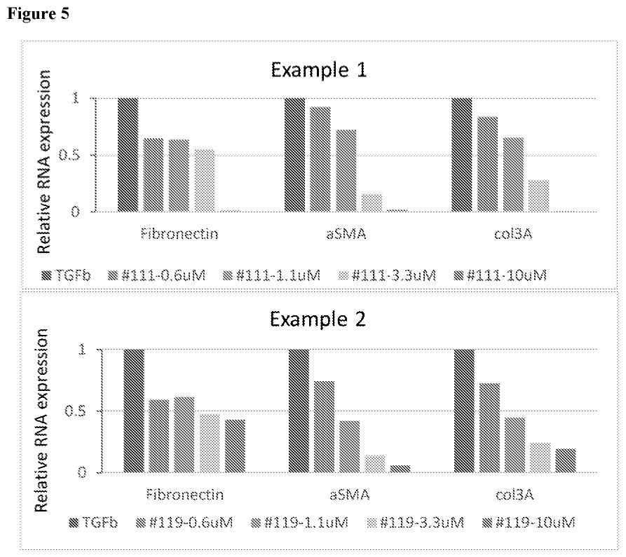

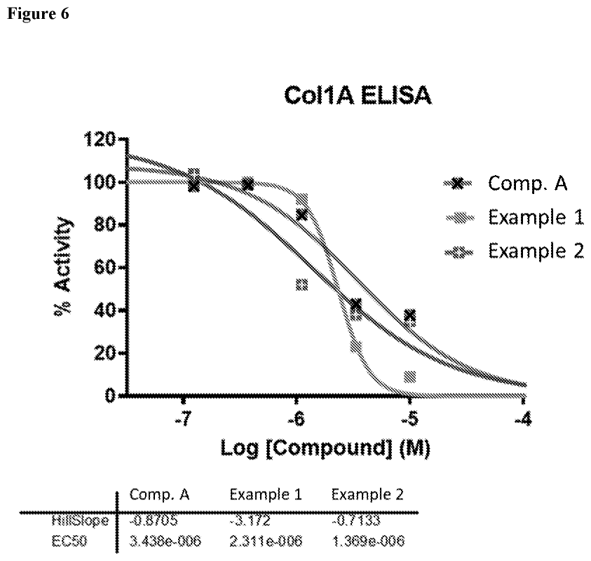

ROCKs are activated by small GTPase Rho in response to a variety of profibrotic signals and regulate cytoskeletal dynamics, activation of different down-stream intracellular targets and expression of key profibrotic genes, such as fibronectin, smooth muscle actin (α-SMA) and collagen 3 (Col3A1). By using MRC-5 human fibroblasts pre-treated with different dosages of selective ROCK2 inhibitors we found that both the compounds of Example 1 and Example 2 down-regulate dose-dependently TGF-β-induced fibronectin, α-SMA, and Col3A1 gene expression determined by PCR (FIG. 5) as well as Col1A protein production in supernatants as measured by ELISA (FIG. 6). These data further confirm that selective ROCK2 inhibition has direct robust anti-fibrotic effect.

In these studies, human lung fibroblast MRC-5 cells were acquired from American Type Culture Collection (ATCC) and cultured in MEM supplemented with 10% fetal bovine serum. The cells were seeded in 24-well cell culture plates at 50,000/mL/well. On the following day, the cells were starved in MEM containing 0.5% FBS for overnight. TGF-β1 (2.5 ng/mL) and various concentration of that the compounds of Example 1 and Example 2 were applied to cells for 48 hours. Cellular mRNA was isolated and mRNA expression of indicated genes (fibronectin, αSMA and col3A) was analyzed by RT-PCR (FIG. 5). Secreted pro-collagen 1a (Col1A1) in supernatants was measured by ELISA (FIG. 6).

### Example 28

### Selective ROCK2 Inhibitors Potently Down-Regulate Differentiation in Human Adipocytes

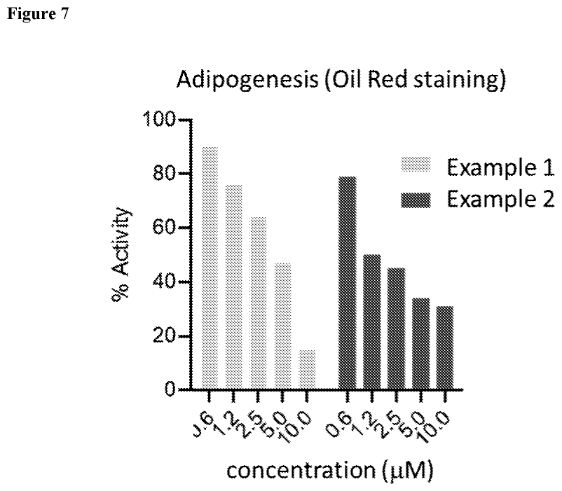

Adipogenesis is a multi-step complex process, progressing from precursor stem cells to adipocytes that synthesize and store fat. The dysregulation of adipocyte function was shown to be involved in pathogenesis of a broad range of metabolic diseases such as type 2 diabetes, obesity, etc. ROCKs have been implicated in regulation of adipocyte differentiation, however the isoform-specific contribution to adipogenesis and other metabolic pathways is not well characterized. Recent data demonstrated that KD025, a selective ROCK2 inhibitor, down-regulated adipocyte differentiation in both mouse 3T3-L1 pre-adipocytes and human adipose-derived stem cells by reducing the expression of key adipogenic/lipogenic genes such as PPARg, C/EBPa and Glut4. By using primary human adipocytes, we found that novel selective ROCK2 inhibitors, Ex. 1 and Ex. 2 down-regulate adipogenesis in a dose-dependent manner (FIG. 7), further confirming the anti-metabolic potential of ROCK2 targeting in cells. In this study, Human subcutaneous adipocytes were grown to confluence before induction to differentiation by adipogenic cocktail (0.1 μM dexamethasone, 1 μM insulin, 200 μM indomethacin, 250 μM isobutyl methylxanthine (IBMX)) in the presence of indicated concentrations of Ex. 1 or Ex. 2 for 10 days. Differentiated cells were stained by Oil Red which was extracted by isopropanol and measured by absorbance at 492 nM.

We claim:

1. A compound having the formula I: 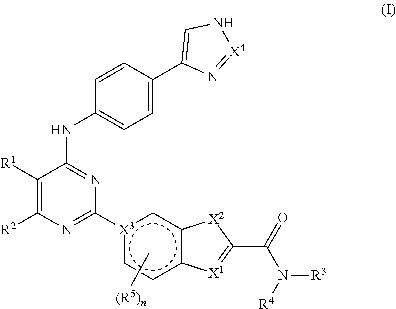
or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
X⁴ is N or CH;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CR⁶, NR⁷, O and S;
X³ is selected from the group consisting of C, CH and N;
each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

2. A compound according to claim 1, having the formula II: 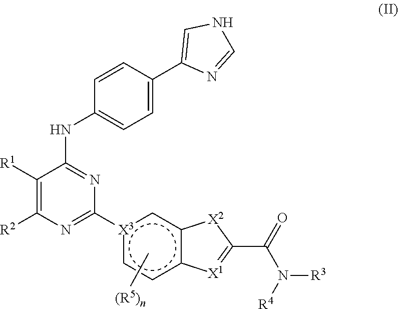
or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CR⁶, NR⁷, O and S;
X³ is selected from the group consisting of C, CH and N;
each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

3. A compound according to claim 1, having the formula IIa: 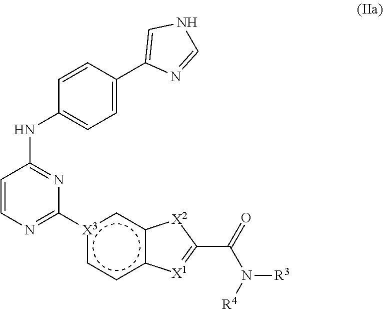
or a pharmaceutically acceptable salt thereof, wherein:
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
X¹ is selected from the group consisting of CR⁶ and N;
X² is selected from the group consisting of CHR⁶, NR⁷, O and S;
X³ is selected from the group consisting of C, CH and N;
each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

4. A compound according to claim 1, having the formula III: 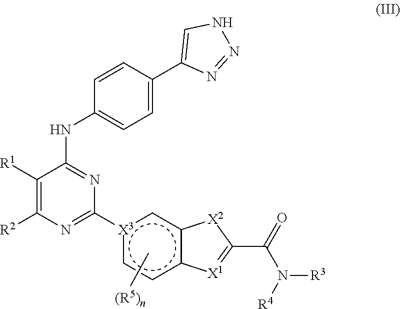
or a pharmaceutically acceptable salt thereof, wherein:
R¹ is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
R² is selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
alternatively, R¹ and R² are taken together to form a 5- or 6-membered saturated or unsaturated fused ring which may contain from 0 to 2 ring heteroatoms selected from the group consisting of N, O, and S, and which is unsubstituted or substituted with 1 to 3 substituents selected from the group consisting of C₁-C₆ alkyl, halo, —CN, —OH, oxo, —O—(C₁-C₆ alkyl), —O—(C₁-C₆ alkyl)-OH, —O—(C₁-C₆ alkyl)-O—(C₁-C₆ alkyl), —NR¹¹R¹², —O—(C₁-C₆ alkyl)-NR¹¹R¹², C₁-C₃ perfluoro alkyl, —NR¹¹—(C₁-C₆ alkyl)NR¹¹R¹², and —NR¹¹—(C₁-C₆ alkyl)-OR¹¹;
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
each R⁵ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, oxo, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
n is 0 to 3;
X¹ is selected from the group consisting of CR⁶ and N;
X² is selected from the group consisting of CR⁶, NR⁷, O and S;
X³ is selected from the group consisting of C, CH and N;
each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

5. A compound according to claim 1, having the formula IIIa: 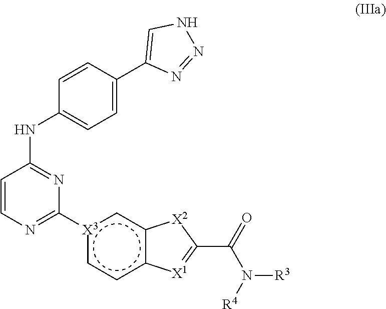
or a pharmaceutically acceptable salt thereof, wherein:
R³ and R⁴ are each independently selected from the group consisting of H, C₁-C₆ alkyl, C₂-C₆ alkenyl, C₂-C₆ alkynyl, C₃-C₇ cycloalkyl, 3- to 10-membered heterocyclyl, C₆-C₁₀ aryl, 5- to 14-membered heteroaryl, C₆₋₁₂ aralkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
alternatively R³ and R⁴ are taken together with the nitrogen to which they are attached to provide (i) a 4- to 6-membered heterocyclic ring having from 0 to 2 additional ring heteroatoms selected from N, O and S, or (ii) a 5- to 10-membered hetero bicyclic ring system having from 0 to 3 additional ring heteroatoms selected from N, O and S; wherein the heterocyclic ring or the hetero bicyclic ring system are unsubstituted or are substituted with from 1 to 4 substituents selected from the group consisting of halo, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, oxo, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
the dotted lines represent optional double bonds;
X¹ is selected from the group consisting of CR⁶ and N;
X² is selected from the group consisting of CR⁶, NR⁷, O and S;
X³ is selected from the group consisting of C, CH and N;
each R⁶ is independently selected from the group consisting of H, C₁-C₆ alkyl, C₂-C₆ alkenyl, C₂-C₆ alkynyl, C₃-C₇ cycloalkyl, halo, —CN, C₁-C₃ perfluoro alkyl, —OR¹¹, —O—(C₁-C₆ alkyl)-OR¹¹, —(C₁-C₆ alkyl)-OR¹¹, —NR¹¹R¹², —O—(C₁-C₆ alkyl)-NR¹¹R¹², —(C₁-C₆ alkyl)-NR¹¹R¹², —NR¹¹—(C₁-C₆ alkyl)-NR¹¹R¹², —NR¹¹—(C₁-C₆ alkyl)-OR¹¹, —(C₁-C₆ alkyl)ₓ-C(═O)R¹¹, —O—(C₁-C₆ alkyl)ₓ-C(═O)R¹¹, —(C₁-C₆ alkyl)ₓ-C(═O)OR¹¹, —C(═O)—R¹¹, —C(═O)OR¹¹, —(C₁-C₆ alkyl)ₓ-C(═O)NR¹¹R¹², —NR¹¹—(C₁-C₆ alkyl)ₓ-C(═O)R¹¹, and —NR¹¹—(C₁-C₆ alkyl)ₓ-C(═O)OR¹¹;
each R⁷ is independently selected from the group consisting of H, C₁-C₆ alkyl, C₂-C₆ alkenyl, C₂-C₆ alkynyl, C₃-C₇ cycloalkyl, —(C₁-C₆ alkyl)-OR¹¹, —(C₁-C₆ alkyl)-NR¹¹R¹², —(C₁-C₆ alkyl)ₓ-C(═O)R¹¹, —(C₁-C₆ alkyl)ₓ-C(═O)OR¹¹, and —(C₁-C₆ alkyl)ₓ-C(═O)NR¹¹R¹²;
each x is independently selected from 0 and 1; and
each R¹¹ and R¹² are independently selected from the group consisting of H and C₁-C₆ alkyl;
or alternatively, R¹¹ and R¹² are taken together when both are attached to the same nitrogen to form a 4- to 7-membered heterocyclic ring having from 0 to 2 additional ring heteroatoms selected from the group consisting of N, O and S, and which heterocyclic ring is unsubstituted or is substituted with 1 to 3 substituents selected from the group consisting of halo, C₁-C₆ alkyl, C₂-C₆ alkenyl, C₂-C₆ alkynyl, C₃-C₇ cycloalkyl, —CN, —NH₂, C₁-C₃ perfluoro alkyl, —OH, —O—(C₁-C₆ alkyl), and —(C₁-C₆ alkyl)-OH.

6. A compound of claim 1, which is 2-[2-(3,3-difluoroazetidine-1-carbonyl)-1-methyl-1H-indol-6-yl]-N-[4-(1H-imidazol-5-yl)phenyl]pyrimidin-4-amine having the formula: 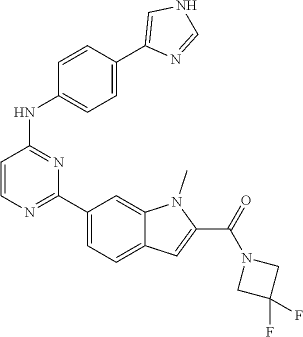 [SMILES: Cn1c(C(=O)N2CC(F)(F)C2)cc2ccc(-c3nccc(Nc4ccc(-c5c[nH]cn5)cc4)n3)cc21]
or a pharmaceutically acceptable salt thereof.

7. A compound of claim 1, which is 2-[2-(3,3-difluoroazetidine-1-carbonyl)-1-methyl-1H-indol-6-yl]-N-[4-(1H-1,2,3-triazol-4-yl)phenyl]pyrimidin-4-amine having the formula: 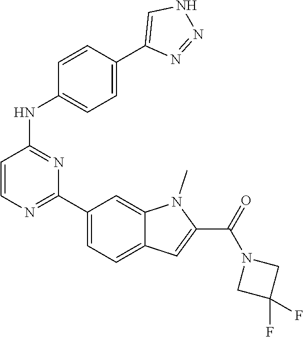 [SMILES: Cn1c(C(=O)N2CC(F)(F)C2)cc2ccc(-c3nccc(Nc4ccc(-c5c[nH]nn5)cc4)n3)cc21]
or a pharmaceutically acceptable salt thereof.

8. A method for the treatment of a disease or disorder mediated by ROCK2, wherein the method comprises administering to a subject in need thereof an effective amount of a compound according to claim 1 or a pharmaceutically acceptable salt thereof.

9. The method of claim 8, wherein the disease or disorder is selected from the group consisting of fibrotic diseases, inflammatory diseases, autoimmune diseases, cardiovascular disorders, central nervous system disorders, neoplastic diseases, metabolic syndromes, ocular diseases, renal diseases, pulmonary diseases, muscular dystrophy, sickle cell disease, and viral diseases.

10. The method of claim 9, wherein the disease or disorder is selected from the group consisting of fibrotic diseases, inflammatory diseases, and autoimmune diseases.

11. The method of claim 10, wherein autoimmune disease is selected from the group consisting of rheumatoid arthritis, systemic lupus erythematosus (SLE; lupus), psoriasis, psoriatic arthritis, multiple sclerosis, Crohn's disease, ulcerative colitis, atopic dermatitis, eczema, or graft-versus-host disease (GVHD; acute and chronic), idiopathic pulmonary fibrosis and scleroderma.

12. The method of claim 9, wherein the disease or disorder is selected from the group consisting of a cardiovascular disorder, a central nervous system disorder, a neoplastic disease, or a metabolic syndrome.

13. The method of claim 10, wherein the inflammatory disorder is selected from the group consisting of cardiovascular inflammation, pulmonary inflammation, renal inflammation, arteriosclerosis and sepsis.

14. The method of claim 10, wherein the fibrotic disorder is selected from the group consisting of idiopathic pulmonary fibrosis, renal fibrosis, kidney fibrosis, ocular fibrosis, cardiac fibrosis, NASH, scleroderma, systemic sclerosis, and cirrhosis.

15. The method of claim 12, wherein the neoplastic disease is selected from the group consisting of ovarian cancer, breast cancer and pancreatic cancer.

16. The method of claim 12, wherein the cardiovascular disease is selected from the group consisting of hypertension, cardiomyopathy, cardiac remodeling, atherosclerosis, restenosis, cardiac hypertrophy, cerebral ischemia, cerebral vasospasm, and erectile dysfunction.

17. The method of claim 9, wherein the pulmonary disease is selected from the group consisting of idiopathic pulmonary fibrosis, chronic obstructive pulmonary disease, and asthma.

18. The method of claim 9, wherein the central nervous system disorder is selected from the group consisting of neuronal degeneration or spinal cord injury, traumatic brain injury, cerebral cavernous malformation, Huntington's disease, Parkinson's disease, Alzheimer's disease, Amyotrophic lateral sclerosis (ALS), and multiple sclerosis.

19. The method of claim 9, wherein the renal disease is selected from the group consisting of polycystic kidney disease, renal fibrosis and diabetic renal disease.

20. The method of claim 9, wherein the metabolic disease is selected from the group consisting of insulin resistance, hyperinsulinemia, type 2 diabetes, obesity, metabolic syndrome and glucose intolerance.

21. The method of claim 9, wherein the ocular disease is selected from the group consisting of ocular hypertension, age related macular degeneration (AMD; wet and dry), choroidal neovascularization (CNV), choroidal tumor, diabetic macular edema (DME), iris neovascularization, uveitis, glaucoma, primary open-angle glaucoma, acute angle-closure glaucoma, pigmentary glaucoma, congenital glaucoma, normal tension glaucoma, secondary glaucoma, neo vascular glaucoma, geographic atrophy, and retinitis of prematurity (ROP).

22. The method of claim 9, wherein the disease is Duchenne muscular dystrophy.

23. The method of claim 9, wherein the viral infection is a coronavirus infections such as SARS-CoV-1, SARS-CoV-2, and MERS-CoV.
